(19) United States
(12) Patent Application Publication
(10) Pub. No.: US 2025/0025651 A1
(43) Pub. Date: Jan. 23, 2025

(54) MASK PROVIDING POSITIVE AIRWAY PRESSURE AND MEASURING CHANGES IN BLOOD PRESSURE

(71) Applicant: KONINKLIJKE PHILIPS N.V., EINDHOVEN (NL)

(72) Inventors: JONAS DEAN COCHRAN, SANTEE, CA (US); MATTHEW BANET, SAN DIEGO, CA (US)

(21) Appl. No.: 18/774,192

(22) Filed: Jul. 16, 2024

Related U.S. Application Data
(60) Provisional application No. 63/528,220, filed on Jul. 21, 2023.

(51) Int. Cl. A61M 16/00; A61M 16/06

(52) U.S. Cl. CPC A61M 16/024; A61M 16/0003; A61M 16/0066; A61M 16/06; A61M 2016/0027; A61M 2205/0216; A61M 2205/0233; A61M 2205/3306; A61M 2205/3327; A61M 2205/3331; A61M 2205/3553; A61M 2205/3561; A61M 2205/3584; A61M 2205/505; A61M 2210/0618; A61M 2230/04; A61M 2230/08; A61M 2230/42; A61M 2230/63

(57) ABSTRACT

The invention provides a wearable mask for monitoring a blood pressure value and providing a flow of gas at a positive pressure to the patient. The mask includes a first sensor, configured to measure a time-dependent optical waveform from a first region underneath a first portion of the wearable mask, with the time-dependent optical waveform including a first pulse. A second sensor measures a time-dependent impedance waveform from a second region proximal to the mask, with the time-dependent impedance waveform including a second pulse. A microprocessor attaches to the wearable mask and is configured to: 1) receive digital representations of both the first and second pulses; 2) process the digital representations to determine a time difference between the first and second pulses, or a parameter calculated therefrom; and 3) process the time difference between the first and second pulses, or the parameter calculated therefrom, to determine the blood pressure value.

### CROSS-REFERENCE TO RELATED APPLICATIONS

This patent application claims the priority benefit under 35 U.S.C. § 119(e) of U.S. Provisional Application No. 63/528,220, filed on Jul. 21, 2023, the contents of which are herein incorporated by reference.

### BACKGROUND AND FIELD OF THE INVENTION

### 1. Field of the Invention

The invention relates to the field of devices that provide positive airway pressure (herein “PAP”) using wearable masks, and to the field of physiological patient monitoring.

### 2. Description of the Related Art

Obstructive sleep apnea (herein “OSA”) is a sleep disorder characterized by repetitive partial or complete blockages of a patient's upper airway during sleep, leading to pauses in breathing. Such pauses, known as apneas, can occur multiple times throughout the night and last for several seconds or even minutes. OSA is often accompanied by disrupted sleep patterns featuring loud snoring, gasping, and/or choking episodes. It affects people of all ages, and is most prevalent in overweight individuals and those over the age of 40. In 2021, roughly 22 million Americans suffered from OSA.

From a physiological perspective, during an apnea event, an upper airway of an OSA patient becomes blocked and breathing is interrupted, resulting in a decrease in the amount of oxygen entering their lungs. This leads to a gradual decline in oxygen saturation (herein “SpO2”) levels. During this period, the patient's blood pressure (herein “BP”) can briefly increase due to the body's response to the lack of oxygen and the resulting activation of the sympathetic nervous system. This response triggers vasoconstriction (narrowing of blood vessels) and an increase in heart rate (herein “HR”), leading to a temporary rise in BP. Concurrently, there is an initial increase in the patient's stroke volume (herein “SV”), which indicates the volume of blood flowing into the aorta with each contraction of the left ventricle. The sympathetic response leads to increased contractility of the heart, resulting in a temporary rise in SV.

As the apnea event progresses and the pause in breathing continues, carbon dioxide (herein “CO₂) levels in the patient's blood increase, triggering a compensatory response. The body's chemoreceptors sense the elevated CO₂ levels and signal the brain to increase respiration rate (herein “RR”). As a result, there is a subsequent drop in BP and an increase in SV due to the vasodilation (widening of blood vessels) that occurs in response to increased CO₂.

Once the apnea event ends and normal breathing resumes, the patient typically experiences a sudden surge in BP attributed to the release of stored stress hormones, such as adrenaline, as the body recovers from the oxygen deprivation experienced during the event. This, in turn, can lead to vasoconstriction, increased HR, and decrease in SV. Cardiac output (herein “CO”, the product of SV×HR), remains relatively constant during this period, as the increase in HR negates the decrease in SV.

Such fluctuations in physiological parameters during apnea—and particularly BP and SV—can strain a patient's cardiovascular system and contribute to the increased risk of disease states hypertension (high blood pressure), congestive heart failure (herein “CHF”), heart attack, stroke, and other cardiovascular diseases.

Known PAP therapies include continuous positive airway pressure (“CPAP”), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping. PAP therapies involve a patient wearing a specialized mask for delivering pressurized air during sleep. A PAP device connects to the mask through a flexible hose, and delivers a constant flow of pressurized air to keep the patient's airway open. The pressurized air prevents the collapse of soft tissues in the throat, allowing for uninterrupted breathing throughout the night. PAP devices typically feature a pressure setting dictated by the severity of the patient's level of OSA; the patient, often with guidance from a healthcare professional, typically adjusts the pressure setting as their PAP therapy is refined. PAP devices are also used for purposes other than treatment of OSA. For example, PAP devices are used to supplement or replace ventilation, treat CHF, stroke, or Cheyne-Stokes respiration.

PAP therapy offers numerous benefits to patients suffering from OSA. The positive air pressure it provides keeps the patient's airway open during sleep, and thus effectively reduces or eliminates their snoring and apnea episodes. Ultimately this leads to improved sleep quality and daytime alertness. PAP therapy helps alleviate symptoms such as excessive daytime sleepiness, morning headaches, and cognitive impairment, and well as ameliorating the deleterious cardiovascular conditions described above that are associated with untreated OSA.

While PAP therapy is highly effective, it does require adherence and adjustment to ensure optimal outcomes. Some individuals may find it initially uncomfortable to wear the mask, or may experience dryness in the nose or throat. However, modern PAP devices are designed to be quieter, smaller, and more user-friendly than their predecessors. Various mask styles and sizes are available to accommodate different preferences and facial structures. Regular follow-ups with healthcare providers are important to monitor progress, address any concerns, and make necessary adjustments to the PAP settings or equipment to ensure optimal therapeutic benefits.

Typical PAP devices use an internal sensor to measure a patient's RR by monitoring flow of air from the patient to the machine. However, these devices lack sensors for measuring other vital signs, such as HR and SpO2, as well as more complex ‘hemodynamic’ parameters, such as SV and CO.

Outside of positive airway pressure devices, there exists many types of patient monitors that operate both in hospitals and at home. For example, such monitors can measure electrocardiogram (herein “ECG”) and impedance pneumography (herein “IPG”) waveforms using torso-worn electrodes, from which they calculate HR, HR variability (herein “HRV”), and RR. Most conventional monitors also measure optical signals, called photoplethysmogram (herein “PPG”) waveforms, with sensors that typically clip on the patient's fingers or earlobes. Algorithms associated with such sensors can calculate SpO2 and pulse rate (herein “PR”) from the PPG waveforms. More advanced monitors can also measure BP, notably systolic (herein “SYS”), diastolic (herein “DIA”), and mean (herein “MAP”) BP. Such measurements are typically made using cuff-based techniques called oscillometry or auscultation, or pressure-sensitive catheters that insert into a patient's arterial system called arterial lines. Digital stethoscopes, which can be either portable and body-worn devices, can measure phonocardiogram (herein “PCG) waveforms that indicate heart sounds and murmurs.

Some patient monitors are entirely body-worn. These typically take the shape of patches that measure ECG, HR, HRV and, in some cases, RR. Such patches can also include accelerometers that measure motion (herein “ACC”) waveforms along x, y, and z-axes. Algorithms can process the ACC waveforms to determine the patient's posture, degree of motion, falls, and other motion-related parameters. Patients typically wear these types of patches in the hospital or, alternatively, for ambulatory and home use. The patches are typically worn for relatively short periods of time (e.g., from a few days to several weeks) and used primarily as cardiac event monitors to detect life-threatening arrhythmias. They typically include wireless transceivers, based on technology such as Bluetooth® or Wi-Fi, to transmit information over a short range to a secondary ‘gateway’ device. The gateway device typically includes a cellular and/or Wi-Fi radio to transmit the information to a cloud-based system.

Even more complex patient monitors measure hemodynamic parameters such as SV, CO, and cardiac wedge pressure using an invasive sensor called a Swan-Ganz or pulmonary-artery catheter. To make a measurement, these sensors are positioned in the patient's left heart, where they are ‘wedged’ into a small pulmonary blood vessel using a balloon catheter. As an alternative to this highly invasive measurement, patient monitors can use non-invasive techniques such as bio-impedance and bio-reactance to measure similar parameters. These methods deploy networks body-worn electrodes (typically deployed on the patient's chest, legs, and/or neck) to measure IPG and/or bio-reactance (herein “BR”) waveforms. Analysis of IPG and BR waveforms yields SV, CO, and thoracic impedance, which is a proxy for fluids disposed in the patient's chest (herein “FLUIDS”). Notably, IPG and BR waveforms generally have similar shapes and are sensed using similar measurement techniques, and are thus used interchangeably herein.

Remote patient monitoring (herein “RPM”) refers to the use of digital technologies to collect and transmit patient health data from patients in their homes or other non-clinical settings to healthcare providers for monitoring and management. Typical RPM patients include those suffering from chronic diseases that often result in frequency hospital readmissions, such as CHF, chronic obstructive pulmonary disease (herein “COPD”), hypertension, and diabetes.

Not surprisingly, there is significant overlap between patients suffering from OSA and these chronic diseases. For example, in 2021, approximately 6.2 million Americans suffered from CHF; estimates suggest that approximately 50-70% of these patients may also have OSA. The link between these conditions is bidirectional, as OSA can contribute to the development and progression of CHF, while CHF can exacerbate the severity of OSA. Similar situations exist for COPD (roughly 10-20% of OSA patients also have COPD), hypertension (30-70%), and diabetes (40-70%). In general, the coexistence of chronic diseases and OSA can have significant implications for patient management. OSA in these individuals may worsen symptoms, increase the risk of cardiovascular or pulmonary events, and impact treatment outcomes. Therefore, proper evaluation and management of OSA in individuals with chronic diseases is crucial for optimizing their overall health and quality of life.

In recent years, RPM has gained significant attention and recognition for its potential to improve healthcare outcomes and reduce costs. The Centers for Medicare and Medicaid Services (herein “CMS”), the federal agency responsible for administering healthcare programs in the United States, has recognized the value of RPM and has implemented reimbursement policies to support its use.

CMS reimburses for remote patient monitoring services under the Medicare program when specific criteria are met. In 2019, CMS introduced new codes and guidelines that allow healthcare providers to bill for RPM services, making it easier for providers to receive reimbursement for monitoring patients remotely. CMS reimbursement typically covers initial setup and patient education, as well as the ongoing monitoring and interpretation of patient data. It includes activities such as the use of connected devices to collect physiological data (e.g., BP, HR) and the time spent by healthcare professionals reviewing and analyzing the transmitted data.

CMS-funded RPM has several benefits. It encourages healthcare providers to adopt and implement RPM technologies, thereby facilitating the remote management of chronic conditions and post-acute care, and reducing the need for in-person visits. This not only improves patient access to care, especially for those in rural or underserved areas, but also enhances patient engagement and empowers individuals to take an active role in managing their own health. By enabling earlier detection of health issues, remote patient monitoring can help prevent complications, reduce hospital readmissions, and ultimately lead to better patient outcomes.

### SUMMARY OF THE INVENTION

Based on the above, it would be beneficial to combine the therapeutic benefit of positive airway pressure (“PAP) therapy, such as CPAP, variable pressure therapy, such as CFlex or BiFlex, or bilevel positive airway pressure therapy, with monitors that measure vital signs and hemodynamic parameters during, e.g., RPM. An ideal system, for example, would feature a positive airway pressure device and mask that collectively deliver therapy to OSA patients while simultaneously measuring vital signs, hemodynamic parameters, and time-dependent physiological waveforms; taken alone or combined, this information can characterize other chronic diseases, such as CHF, COPD, hypertension, and diabetes. Additionally, providing these types of data may further engage the patient in their PAP therapy, thereby improving compliance. This, in turn, increases the efficacy of the PAP therapy. Additionally, including patient-monitoring capabilities directly within the PAP mask may allow determination of parameters such as arousals during sleep, apnea events, improper mask fitting, mask deterioration, and other physiological and mask-related parameters. It may also indicate a mask that is does not fit the patient properly, is leaking, or needs replacement. Knowledge of these issues, in turn, may allow clinicians to adjust the patient's PAP therapy, thereby increasing its efficacy and improving the patient health.

The current invention effectively accomplishes these objectives with an ensemble of embedded, electronic sensors coupled to a control system, with each of these components integrated directly within the PAP mask. The control system provides power and includes analog and digital electronics that, during use, process signals that the sensors measure to determine the vital signs, hemodynamic parameters, and time-dependent waveforms. It also includes data-transmission systems that send both raw and processed information to external patient-management systems. Ultimately this helps connect OSA patient to their clinicians. Taken in combination, these systems provide the patient with PAP therapy while simultaneously allowing clinicians to remotely monitor patients, e.g., as per RPM.

More specifically, the invention provides a ‘Smart Mask’ (herein “SMK”) that includes one or more of the following sensors, each embedded directly into the mask and configured to measure signals directly from (or proximal to) a patient's face: 1) an optical sensor for measuring PPG waveforms and, from these, SpO2 and PR; 2) an impedance sensor for measuring IPG/BR waveforms and, from these, SV, CO, and FLUIDS; 3) an EMG sensor for measuring muscle activity from the patient's face and EMG waveforms and, from these, arousal-related muscle activity; 4) a flow sensor for measuring breathing rate, temperature, humidity, and volatile organic compounds (herein “VOCs”) and volatile sulfur-containing compounds (herein “VSCs”) from the patient's breath and, from these, RR; 5) a digital microphone for measuring sounds emitted by the patient, e.g., snoring, wheezing, and coughing; and 6) an accelerometer for measuring ACC waveforms along x, y, and z-axes, and from these determining the patient's posture and degree of motion.

Additionally, algorithms operating on a microprocessor within the SMK can further analyze the PPG, ECG, and IPG/BR waveforms to determine SYS, MAP, or DIA BP, as described in more detail below. In embodiments, a battery-powered control system located directly on the SMK controls each of the above-mentioned sensors to make their corresponding measurements. Wired and wireless transmitters in the control system transmit information to an external gateway, which then forwards it to the cloud. There, algorithms based on ML and AI process the SMK-measured information to determine parameters such as arousal, apnea events, physiological information used for RPM, how well the SMK fits the patient, if the SMK needs replacement, along with other parameters. By providing this information, the SMK provides PAP therapy while simultaneously monitoring the patient. It helps inform remote clinicians of the patient's physiological condition and how well the PAP therapy is working. Ultimately, this information may help connect and inspire dialogue between clinicians and the patient, and by doing so improve the patient's compliance with the PAP therapy.

In one aspect, the invention provides a system for monitoring a BP value from a patient. The system features a wearable mask (e.g., the SMK) coupled to a PAP machine that delivers air at positive pressures to the patient. The wearable mask includes a first sensor connected directly thereto, e.g., an optical sensor configured to measure a time-dependent optical waveform (e.g., PPG waveform) from a first region underneath a first portion of the wearable mask, with the time-dependent optical waveform including a first pulse. The mask also includes a second sensor connected directly thereto, e.g., an impedance sensor that measures a time-dependent impedance waveform (e.g., IPG and/or BR waveforms) from a second region proximal to the wearable mask, with the time-dependent impedance waveform including a second pulse. A processing system featuring a microprocessor attaches to the wearable mask and is configured to: 1) receive digital representations of both the first and second pulses; 2) process the digital representations to determine a time difference between the first and second pulses, or a parameter calculated therefrom; and 3) process the time difference between the first and second pulses, or the parameter calculated therefrom, to determine the BP value.

In embodiments, the optical sensor includes both first and second light sources and a photodiode, wherein the first light source can be a first light-emitting diode (herein “LED”) that emits optical radiation near λ=660 nm, and the second light source can be an LED that emits optical radiation near λ=940 nm. By “near” it is meant that the LED emits radiation within about +20 nm of the target emission wavelength.

In embodiments, the impedance sensor includes at least one sense electrode and at least one drive electrode. Both the electrodes typically feature a conductive material selected from the following: rubber, polymer, fabric, metal, wire, mesh, and hydrogel; other conductive materials may also be used. The drive electrode is configured to inject a first electrical current into the second region, wherein the current is modulated at a frequency ranging from 5-500 kHz and has an amplitude ranging from 0.01-5 mA. In embodiments, the first sense electrode and drive electrode connect to a first side of the wearable mask. Typically, on an opposing side, the SMK includes a second sense and drive electrode, connected to a second side. Here, the second drive electrode injects electrical current modulated and having an amplitude similar to that injected by the first drive electrode, with the second electrical current modulated at a frequency that is approximately 90° out of phase with the frequency corresponding to the first electrical current.

In embodiments, to determine BP values, the processing system determines a ‘foot’ of the first pulse and a foot of the second pulse, where the foot is located near a minimum amplitude of the pulse, just before it begins to rapidly increase in amplitude. Typically to determine the time difference between the first and second pulses, the processing system calculates a temporal separation between at least one of the following features (also called ‘fiducials’) of the first and second pulses: foot, maximum slope of the pulse's rising edge, base, peak value, and rising edge. The processing system then calculates an inverse of the temporal difference along with a calibration value to determine the BP value. In embodiments, algorithms may process the digitized pulse to help identify the above-mentioned fiducials. Such algorithms include digital filtering, Fourier analysis (or related frequency-domain transforms), smoothing, and/or taking one or more derivatives of the pulse. The derivatives may be single point or multi-point derivatives.

In other embodiments, the wearable mask includes a control system, typically integrated directly into it and, in embodiments, located near its top portion. The control system includes the processing system. The microprocessor is within the processing system, and operates computer code that determines the blood pressure value. The computer code can be written in one or more languages, e.g., C, C++, assembly, Java, Java Script, and Python. A rechargeable battery powers the system, which electrically connects to the first and second sensors through a flexible electrical cable that runs alongside the mask.

In another aspect, the invention provides another type of mask that performs the above-described functions. For example, in addition to providing CPAP therapy, the mask could be used with a variable pressure device (e.g., BiPAP®, Bi-Flex®, or C-Flex™ devices manufactured and distributed by Philips Respironics of Murrysville, Pennsylvania. Or the mask could be one used for a ventilator, oxygen mask, fighter pilot's mask, eye mask, facial wrap, scuba mask, decorative mask (such as a mask used for Halloween), or other type of mask worn near the face.

In another aspect, the invention provides a system for monitoring a BP value from a patient, featuring a wearable mask (e.g., the SMK) that includes an optical sensor and an electrical impedance sensor, with both sensors contacting the patient's face. The optical sensor measures a first time-dependent optical waveform (e.g., PPG waveform) including a first pulse, and the impedance sensor measures a second time-dependent impedance waveform (e.g., an IPG waveform) including a second pulse. The processing system processes a time difference between the first and second pulses, or a parameter calculated therefrom, to determine the BP value.

These and other advantages of the invention should be apparent from the following detailed description, and from the claims.

### BRIEF DESCRIPTION OF THE DRAWINGS

FIG. 1A is a schematic drawing showing a front view of the SMK according to the invention;

FIG. 1B is a photograph of the SMK of FIG. 1A:

FIG. 2 is a schematic drawing of a patient wearing the SMK of FIG. 1A and 1B as it sends information (e.g., vital signs, hemodynamic parameters, and time-dependent waveforms) measured from the patient through a gateway device and/or mobile device to a cloud-based software system, and from there to a 3^{rd}-party software system;

FIG. 3A. 3B, and 3C are schematic drawings of, respectively, a reflective optical sensor, a 4-electrode impedance sensor, and a pressure sensor incorporated in the SMK of FIG. 1A and 1B to measure signals from the patient;

FIG. 3D is a schematic drawing of a transmissive, multi-wavelength optical spectrometer incorporated into a tube connecting a PAP device to the SMK and configured to measure an optical spectrum of the patient's breath within the tube;

FIG. 3E is a photograph of the sensors of FIG. 3A, 3B, and 3C;

FIG. 4A and 4B are top views of, respectively, a front and back surface of the printed circuit board (herein “PCB”) and electrical components making up the control system within the SMK of FIG. 1A and 1B;

FIG. 5A and 5B are, respectively, mechanical drawings showing top and side views of the control system of the SMK of FIG. 1A and 1B near a swivel joint that connects the SMK to a PAP device;

FIG. 5C and 5D are photographs of, respectively, the PCB within the control system of FIG. 4A and 4B integrated into an enclosure within the SMK, and the PCB outside of the enclosure;

FIG. 6A-6F are graphs of time-dependent waveforms measured from the patient by the SMK and featuring, respectively, an ECG waveform (FIG. 6A), PPG waveform (FIG. 6B), IPG waveform (FIG. 6C), pressure waveform (FIG. 6D), EMG waveform (FIG. 6E), and an ACC waveform (FIG. 6F);

FIG. 6G is a schematic drawing showing placement of the SMK used to measure the time-dependent waveforms shown in FIG. 6E-6F;

FIG. 7A and 7B are graphs of time-dependent IPG waveforms measured, respectively, from the face of a patient using the SMK, and from the chest of a patient using a body-worn patch;

FIG. 7C and 7D are schematic drawings showing placements of, respectively, the SMK used to measure the IPG waveform of FIG. 7A, and the body-worn patch used to measure the IPG waveform of FIG. 7B;

FIG. 8A and 8C are graphs of, respectively, a time-dependent PPG waveform measured from a patient's wrist with an optical sensor within a conventional ‘smart watch’, and a time-dependent pressure waveform measured simultaneously from the patient's face with the pressure sensor within the SMK during periods of normal breathing, wheezing, coughing, and apnea;

FIG. 8B and 8D are schematic drawings showing placements of, respectively, an optical sensor and pressure sensor used to measure the time-dependent waveforms of FIG. 7A and 7C;

FIG. 9 is a table showing values of accuracy and area under the curve (herein “AUC”) corresponding to different machine learning (herein “ML”) models used to process data similar to that generated by the SMK and used to determine arousals in OSA patients;

FIG. 10 is a receiver operating characteristic (herein “ROC”) graph plotting a true positive rate vs. a false positive rate for data processed with model 8 in the table shown in FIG. 9;

FIG. 11A and 11B are mechanical drawings of, respectively, side and top views of the bedside hub that connects to the SMK to charge its internal battery and download data stored on its internal flash memory for display and analysis;

FIG. 11C is a photograph of a bedside hub indicated by the mechanical drawings in FIG. 11A and 11B connected to the SMK and a PAP machine;

FIG. 12A-F are screen captures of a graphical user interface (herein “GUI”) that operates on the bedside hub and displays, respectively, a standard clock (FIG. 12A), a first portion of a survey for an OSA patient wearing the SMK (12B), a time-dependent ECG waveform measured by the SMK (FIG. 12C), a second portion of the survey (12D), a third portion of the survey and an indication that files containing physiological data have been successfully uploaded to the cloud (12E), and a time-dependent IPG waveform measured by the SMK (FIG. 12F);

FIG. 13A is a schematic drawing showing electrode locations on a patient wearing the SMK for measuring ECG waveforms according to an alternate embodiment of the invention;

FIG. 13B is a time-dependent ECG waveform measured from a patient according to the electrode locations shown in FIG. 13A and then sent wirelessly to a mobile device, where it is rendered on a GUI;

FIG. 14A is a schematic drawing of an alternate embodiment of the invention featuring a transmissive, multi-wavelength optical spectrometer incorporated into a hose connecting a PAP device to the SMK and measuring an optical spectrum of the patient's breath within the hose;

FIG. 14B is a photograph of a chip-level multi-wavelength optical spectrometer (the ASM AS7341) used in the alternate embodiment of the invention shown in FIG. 14A;

FIG. 14C is a graph of a series of frequency-dependent measurement bands, each activated with a different software register used to control the chip-level multi-wavelength optical spectrometer shown in FIG. 14B; and

FIG. 15 is a graph showing frequency-dependent absorption spectra measured from a sample using a conventional optical spectrometer (Thorlabs, shown on the vertical axis on the left-hand side of the graph) and the chip-level multi-wavelength optical spectrometer of FIG. 14B (AS7341, shown on the right-hand side of the graph).

### DETAILED DESCRIPTION OF THE INVENTION

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

### 1. System Overview

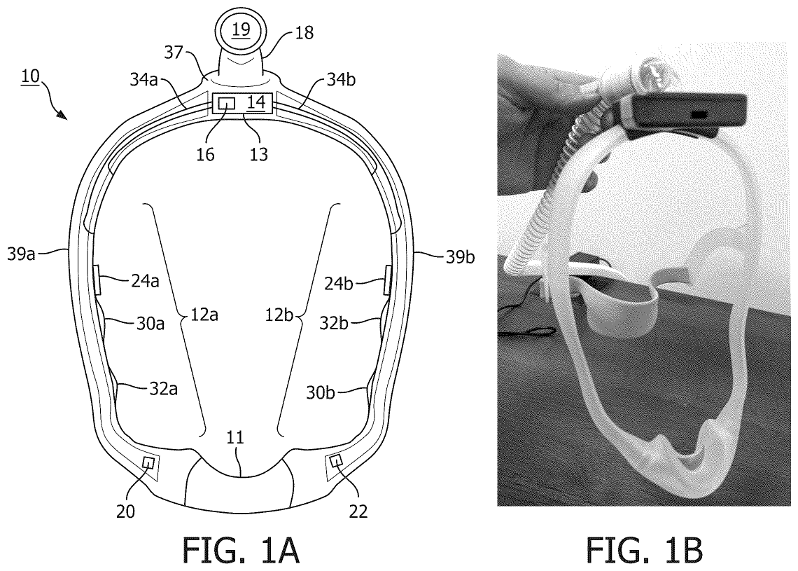

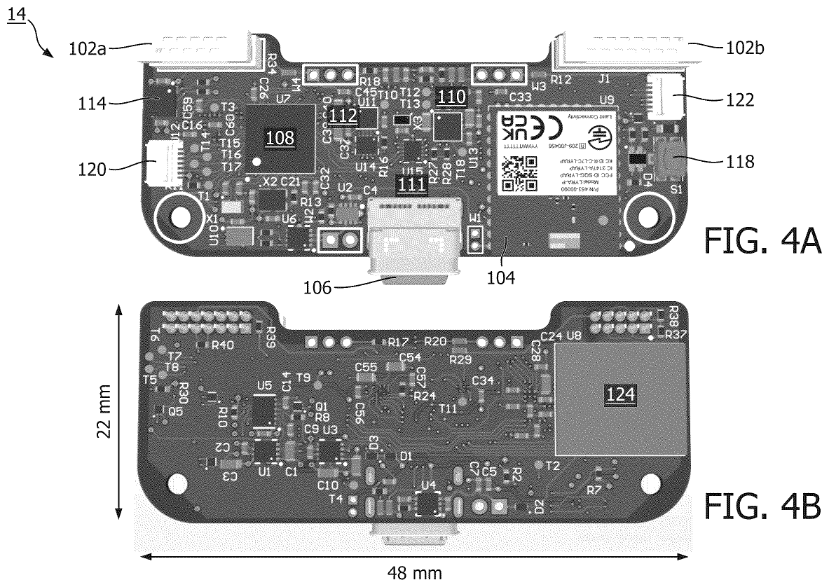

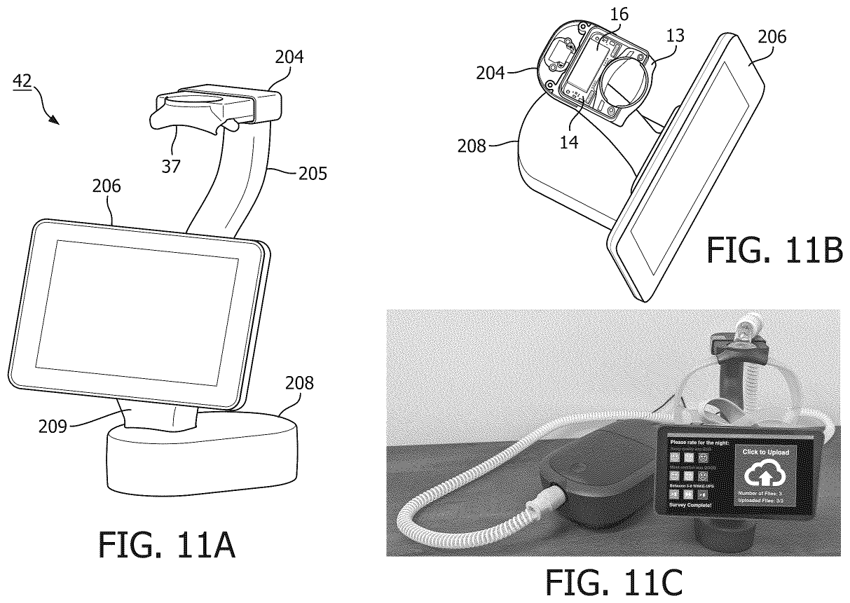

FIG. 1A and 1B show, respectively, a mechanical drawing and photograph of a SMK 10 according to the invention that measures vital signs, hemodynamic parameters, and time-dependent waveforms from a patient using an ensemble 12a, 12b of sensors. A first portion of the ensemble of embedded sensors 12a is located on the left-hand side of the SMK 10; the second portion 12b on the right-hand side. A control system 14 located near a top portion 37 of the SMK 10, and composed primarily of a PCB shown in FIG. 4A and 4B, is enclosed in a mechanical housing 13 that wraps around a swivel connector 18. A rechargeable lithium-ion (herein “Li:ion”) battery 16 powers the SMK 10. The swivel connector 18 includes an opening 19 that connects the SMK 10 to a flexible hose that, in turn, feeds positively pressurized air generated by a remote PAP device (shown, for example, in FIG. 11C) through the SMK 10 and to the patient.

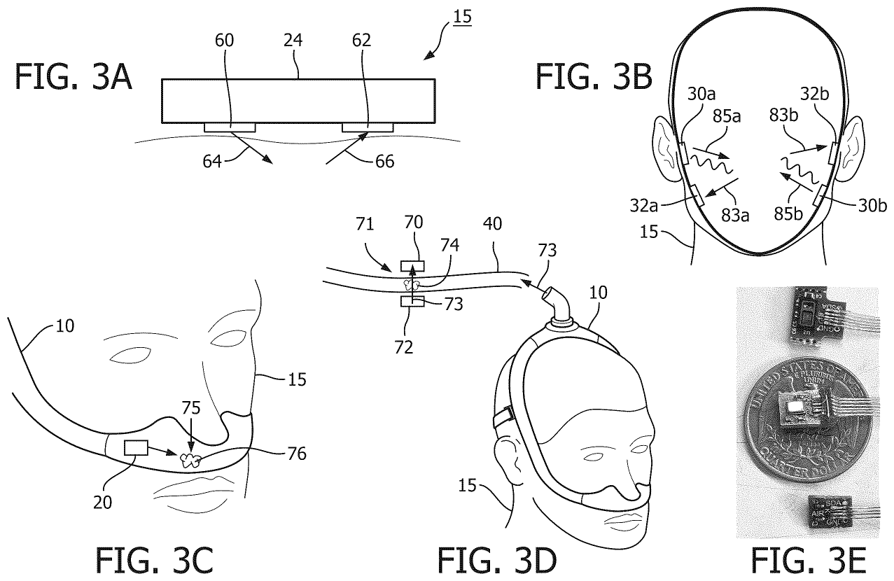

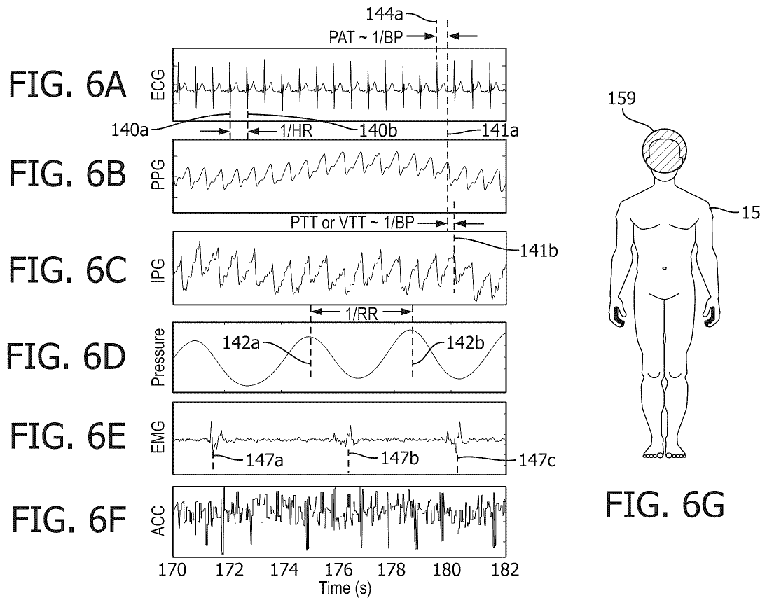

Collectively, sensors within the SMK 10 measure time-dependent waveforms, such as those shown in FIG. 6A-6F, from the patient's face and cheek. To do this, flexible ribbon cables 34a, 34b containing a collection of conductive wires encased in a silicone cladding connect to side portions of the control system 14. Photographs of the control system and sensors shown in FIG. 3E, 5C, and 5D show the ribbon cables in more detail. They span the length of the left 39a and right-hand 39b tubes of the SMK 10 and electrically connect to sensors disposed along these tubes. The SMK's left 39a and right-hand 39b tubes are typically silicone structures that are flat, flexible, and hollow; they connect to a ‘cushion’ or ‘mouthpiece’ component 11 that attaches to the patient's mouth and nose during sleep to supply positively pressurized air.

A pair of reflective optical sensors 24a, 24b disposed on the left 39a and right-hand 39b tubes measure time-dependent PPG waveforms from the patient's cheek, an area featuring dense capillary beds and thus particularly well-suited for this measurement. The first optical sensor 24a connects to conductive wires in the ribbon cable 34a on the left-hand side of the mask; the second optical sensor 24b connects to conductive wires in the ribbon cable 34b on the right-hand side. Referring to FIG. 1A and 3A, each optical sensor 24a, 24b includes a light source 60, which typically features one or more LEDs or, alternatively, diode lasers. The light source 60 typically includes a first LED operating in the red spectral region (e.g., λ=660 nm), and a second LED operating in the infrared spectral region (e.g., λ=940 nm); such wavelengths are ideal for SpO2 measurements, as is known in the art.

During a measurement, the separate LEDs within the light source sequentially emit beams of radiation (schematically indicated by the arrow 64) into the patient's flesh, where they are partially absorbed by blood-filled capillaries therein. The capillaries expand and contract with pulsatile blood flow driven by each of the patient's heartbeats, thereby modulating absorbance of the incident red and infrared radiation according to Beer's Law (which depends on the optical pathway of the incident radiation, and is thus related to the vessel's diameter). After passing through the capillaries, a portion of the modulated radiation (schematically indicated by the arrow 66) irradiates a photodetector 62, where it is optically absorbed to generate a photocurrent that is further processed (e.g., filtered and then amplified) to yield PPG waveforms corresponding independently to the red and infrared spectral regions.

Typically, to collect these waveforms, the control system 14 includes a chip-level analog front end (herein “AFE”, such as Analog Devices MAX86176) that connects to both the light source 60 and photodetector 62 and controls them to sequentially emit red and infrared radiation, and then measure the corresponding PPG waveforms. Notably the MAX86176 includes separate AFEs to measure both PPG and ECG waveforms. Algorithms operating on a microprocessor (herein “CPU”) within the control system 14 receives and processes digital versions of the waveforms, and use digital signal processing to generate signal components representing high-frequency pulsatile PPG elements (typically referred to as ‘RED_{AC}’ and ‘IR_{AC}’) and low-frequency static PPG elements (typically referred to as ‘RED_{DC}’ and ‘IR_{DC}’). This processing ultimately yields SpO2 and PR values, as is known in the art.

The optical sensors 24a, 24b disposed on each side of the SMK 10 provide redundancy and increase the probability that the SMK 10 measures valid PPG waveforms, along with SpO2 and PR values, from the patient. For example, both optical sensors 24a, 24b will likely make good contact with the patient's face and cheeks, and thus yield good measurements, when the patient sleeps on their back with their head upright and undergoes minimal motion. In embodiments, measurements of SpO2 and PR taken from the left and right cheeks can be averaged together to increase measurement accuracy. However, for a patient sleeping on their side or undergoing motion, one optical sensor (e.g., the one between their cheek and the pillow) may make good contact with the patient's cheek and yield good measurements, while the opposing sensor (e.g., the one facing upright) may make poor contact and yield sub-par measurements. In this configuration, the optical sensor yielding good measurements is ideal for characterizing the patient. Such an embodiment requires the CPU to run algorithms that evaluate the PPG waveforms to determine their signal quality.

In still other embodiments, heartbeat-induced pulses in PPG waveforms measured from the left and right cheeks may feature a temporal difference between their pulsatile features. Typically, the temporal difference is measured from the base of each pulsatile feature, and is referred to as pulse arrival time (herein “PAT”), pulse transit time (herein “PTT”) or, alternatively, vascular transit time (herein “VTT”). Previous research indicates that PAT, PTT and VTT are indirectly related to BP, e.g., SYS, MAP, and DIA. FIG. 6A-6C show these temporal components, which are also known as ‘systolic time intervals’, in more detail.

The relationship between PAT, PTT, VTT and BP is influenced by various factors, including arterial compliance, distance between measurement sites, and stiffness of the patient's arterial walls. A linear equation describes this relationship.

$$B P = {m}_{patient - specific} \times \frac{1}{( P A T , P T T , V T T )} + B {P}_{C a l}$$

where m_{patient-specific} is a patient-specific slope describing the relationship between the inverse of PAT, PTT, and/or VTT and BP, and BP_{cal} is an initial calibration value. This equation is the same for SYS, DIA, and MAP, with the equation's constants (m_{patient-specific} and BP_{cal}) depending on what specific BP value is being measured.

In related embodiments, the presence of two optical sensors 24a, 24b means the SMK can simultaneously measure PPG waveforms from both sides of the patient's face. Temporal differences in heartbeat-induced pulses within these waveforms may indicate certain aspects of the patient's physiology. Such a measurement is comparable to that described in U.S. Pat. Nos. 7,803,120 and 9,622,710 to Banet et. al, both of which describe a measurement of ‘bilateral pulse transit time’ (herein “BPTT”), and are incorporated herein by reference. In these documents, BPTT measurements are made using optical sensors disposed on a patient's fingers. They describe how in a BPTT measurement, the asymmetric position of the heart, coupled with the assumption that blood pressure is equivalent along the left and right-hand vascular pathlengths in the body, means the PTT for the right-hand pathlength may be slightly longer than the PTT for the left-hand pathlength. When used with a calibration measurement, this time difference—the BPTT, a systolic time interval similar to PAT, PTT, and VTT—can be used to estimate BP, e.g., SYS, MAP, and DIA.

Typically, the time difference for BPTT measurements is similar to that for PAT, PTT, and VTT, e.g., between about 10-200 ms. Examples where the BPTT is exceedingly long, e.g., >250 ms, or the shapes of heartbeat-induced pulses measured with the different optical sensors are significantly different, may indicate that arteries (e.g., carotid arteries) on one side of the patient's neck and/or face differ from those on the other. Such a difference may occur, for example, if there is significant plaque build-up on one artery compared to the other. In this way, the SMK may be used as a screening tool to estimate if the patient has significant plaque build-up in one of their carotid arteries.

Referring to both FIG. 1A and 3B, electrodes 30a, 30b, 32a, 32b are typically composed of a flexible conductive material (e.g., conductive rubber, fabric, mesh, or textiles) and connect to conductive wires within the ribbon cables 34a, 34b and measure weak, bio-electric signals during sleep. The body's impedance changes over time as the heart contracts and relaxes; this, in turn, modulates the bio-electric signals. More specifically, during systole (when the heart contracts), the IPG waveform shows an initial rise in impedance due to the decrease in thoracic blood volume. This rise is followed by a rapid decline as blood is ejected into the systemic circulation. In diastole (when the heart relaxes), the waveform displays a gradual increase in impedance as blood returns to the thorax.

To measure IPG waveforms from a patient 15, the SMK 10 includes a pair of ‘sense’ electrodes 30a, 30b, and a pair of ‘drive’ electrodes 32a, 32b that, collectively, perform bio-impedance and bio-reactance measurements. The drive electrodes 32a, 32b inject high-frequency (e.g., 5-500 kHz, and typically about 70 kHz), low-amperage (e.g., 0.1-4 mA, and typically about 1 mA) current into the patient's cheek; this is indicated in FIG. 3B by arrows 85a, 85b. As indicated in the figure, current injected by each drive electrode typically features a sinusoid (or, alternatively, square-wave) profile, and is 90° out of phase. The sense electrodes 30a, 30b detect weak bio-electrical signals from each cheek; this is indicated in the figure by arrows 83a, 83b. The bio-electrical impedance is measured by determining the resistance (i.e. opposition to the flow of injected current) and reactance (ability to store and release electrical energy generated by the injected current) in this tissue. Analyzing voltage changes that occur when the electrical current passes through the cheek yields bio-impedance and bio-reactance values that manifests in the form of time-dependent waveforms (e.g., IPG and BR waveforms). As with the optical measurement described above, bio-impedance and bio-reactance measurements are typically managed with a chip-level AFE (e.g., the Analog Devices MAX30009) that is disposed on the control system 14.

In embodiments, the electrodes are conductive fabrics or textiles integrated directly into (e.g., sewn into) the material that attaches the flexible ribbon cables to the mask's tubes that supply pressurized air to the patient.

BR waveforms are a type of IPG waveforms that have a similar morphology, but represent time-dependent phase changes induced by physiological events (e.g., heartbeat-induced blood flow) in tissue disposed underneath the electrodes. Such changes are due to electrical capacitive and inductive properties in the tissue, which are typically weaker than the changes in intrathoracic volume that yield IPG waveforms. BR waveforms may have some improvements over IPG waveforms, mostly in that they are less sensitive to interferences by noise and external sources, and feature a higher percentage of AC signal components relative to DC signal components, as is described in more detail below.

Impedance-measuring systems like the MAX30009 measure a time-dependent impedance waveform that, much like the above-described PPG waveforms, feature an AC component (i.e. a pulsatile component) and a DC component (i.e. a baseline). Algorithms can collectively process the AC and DC components to determine SV and CO, and the DC component alone to estimate FLUID in the patient. SV, CO, and FLUID represent hemodynamic parameters and can be good predictors of chronic diseases, particularly CHF. Examples of such an algorithms are the Sramek-Bernstein and Kubicek equations, both of which estimate SV based on the relationship between changes in thoracic impedance and the rate of change of blood volume during systole. Both equations are described in detail in U.S. Pat. No. 11,129,537 to Banet et. al, the contents of which are incorporated herein by reference.

In most cases, ECG waveforms cannot be measured with good signal-to-noise ratios from locations above the patient's neck, e.g., the face. Thus, the sense electrodes 30a, 30b typically do not yield ECG waveforms that are adequate to calculate HR, RR, etc. This is because the bio-potential signals measured with electrodes on opposite cheeks are exceedingly similar, and thus when processed with a differential amplifier (e.g., that within the ECG AFE in the MAX86176) present in conventional ECG circuits, the resultant signal has essentially no amplitude. However, a differential amplifier similar to that used in ECG circuits can process signals measured by the sense electrodes 30a, 30b to yield a time-dependent waveform similar to ECG called an electromyography (herein “EMG”) waveform that indicates electrical activity produced by skeletal muscles. FIG. 6E shows an example of such an EMG waveform, as measured with the MAX86176. Such activity, which manifests itself in the EMG waveform as a time-dependent pulse, typically indicates that muscles in the patient's face are moving during sleep (e.g., the patient is clenching their jaw), and may be incorporated into a numerical algorithm that estimates arousal. Here, analog filters built into the AFEs for impedance, ECG (and by proxy EMG) measurements are designed to process the bio-electric signals that the sense electrodes 30a, 30b measure to simultaneously yield IPG, EMG, and ECG waveforms.

Referring to FIG. 1A and 3C, to measure time-dependent pressure within the SMK 10, and more specifically the mask's left 39a and right-hand 39b tubes, the flexible ribbon cable 34a on the left-hand side of the SMK 10 connects to a pressure sensor 20 (e.g., the Bosch BME688) disposed near the cushion component 11. Typically, the pressure sensor 20 includes a small opening that detects breath expelled by the patient 15, as indicated schematically by the arrow 75 and ‘cloud’ graphic 76 in FIG. 3D. A porous membrane (not shown in the figure) may be used to cover the opening to prevent it from contaminating the airway. From the breath, the pressure sensor 20 detects time-dependent pressure variations in the mask that are modulated by the patient's breathing pattern. Analysis of these variations yields the patient's RR. Additionally, the BME688 sensor can also detect other parameters that may indicate decompensation of the patient; these include humidity, breath temperature, VOCs, and VSCs.

FIG. 3E shows a photograph of the sensors within the SMK that are described above. The top, middle, and bottom portions of the photograph show, respectively, the optical sensor of FIG. 3A, the digital microphone, and the pressure sensor of FIG. 3C.

In related applications, the pressure sensor 20 can be coupled with an optical sensor (e.g., an infrared optical sensor) that measures concentrations of carbon dioxide (herein “CO₂”) in the patient's breath. For example, to make this measurement, the pressure sensor 20 shown in FIG. 3D can be coupled with the reflective optical sensor 24 shown in FIG. 3A; here, the LED (or, alternatively, laser diode) within the optical sensor emits optical radiation at wavelengths that are strongly absorbed by CO₂, e.g., 2,2.9, and 4.3 microns. Concentration of CO₂ in the patient's breath modulates optical absorption at these wavelengths, which when coupled with a calibration (e.g., one done during manufacturing of the SMK) can yield absolute values of this gas. When used in this application, the combined system—which includes both pressure and optical measurements—effectively functions as a capnography sensor, which provides the concentration or partial pressure of CO₂ within respiratory gases emitted by the patient. In this way, the SMK 10 functions similarly to a sensor that measures respiratory tidal volumes and end-tidal CO₂ (herein “et-CO₂”) a measurement typically reserved for hospitalized patients in the intensive care unit (herein “ICU”). With the SMK, this measurement can be performed at home.

Recent research as shows that such measurements of et-CO₂ and, particularly, their variation can be good predictors of brain natriuretic peptide (herein “BNP”), a known reliable marker for decompensation in patients with CHF (see, e.g., Koyama et. al, “Technology Applications of Capnography Waveform Analytics for Evaluation of Heart Failure Severity”, J Cardiovasc Transl Res. 2020 December; 13 (6): 1044-1054. doi: 10.1007/s12265-020-10032-5. Epub 2020 May 28. PMID: 32462611). BNP is a hormone produced by the ventricles of the heart in response to stretching of heart muscle cells, and plays a crucial role in regulating BP and fluid balance. BNP levels typically increase when there is increased stress on the heart, such as during CHF. Consequently, BNP has been widely studied as a predictor and diagnostic tool for characterizing this chronic condition.

In preferred embodiments, and as described above, the pressure sensor is the Bosch BME688 sensor, or something equivalent. Such a sensor is configured to measure the following parameters from the patient's breath: pressure, temperature, humidity, VOCs, and VSCs. The sensor, taken alone or coupled with complementary sensors, can also measure other parameters such as alcohol content, ketones (e.g., acetone and acetoacetone), glucose levels, and other chemicals in the patient's breath.

The SMK 10 near the cushion 11 can also include a digital microphone 22 that connects to the flexible ribbon cables 34a, 34b and measures sounds emitted from the patient during sleep, e.g., snoring, coughing, wheezing, and apnea events. When sampled at high rates (e.g., several kHz), the digital microphone 22 can measure full-resolution sounds. Lower sample rates (e.g., 100-500 Hz), which are often desirable to reduce the amount of memory required in the SMK 10, yield downsampled signals that serve as approximations for these sounds. Similar to the pressure sensor, the microphone may be covered with a film or porous membrane to keep it from contaminating the patient's airway.

While FIG. 1A and 1B show sensors integrated to a particular type of PAP mask, in theory they can be integrated with any PAP mask, regardless of size or shape. This may require a specialized, mask-specific adaptor that connects the various sensors described above to flexible components in the mask. For example, the sensors may be integrated with the following PAP masks manufactured by Philips/Respironics: Dreamware, DreamWisp, Wisp, ComfortGel, Amara, Pico, Nuance, ComfortGel. Such masks may be full face masks, or dedicated nasal or mouth PAP systems. It should also be noted that the sensors can be integrated into any other mask that provides similar functions as a PAP mask, such as a mask used to provide ventilation to the patient using, e.g., a non-invasive ventilator.

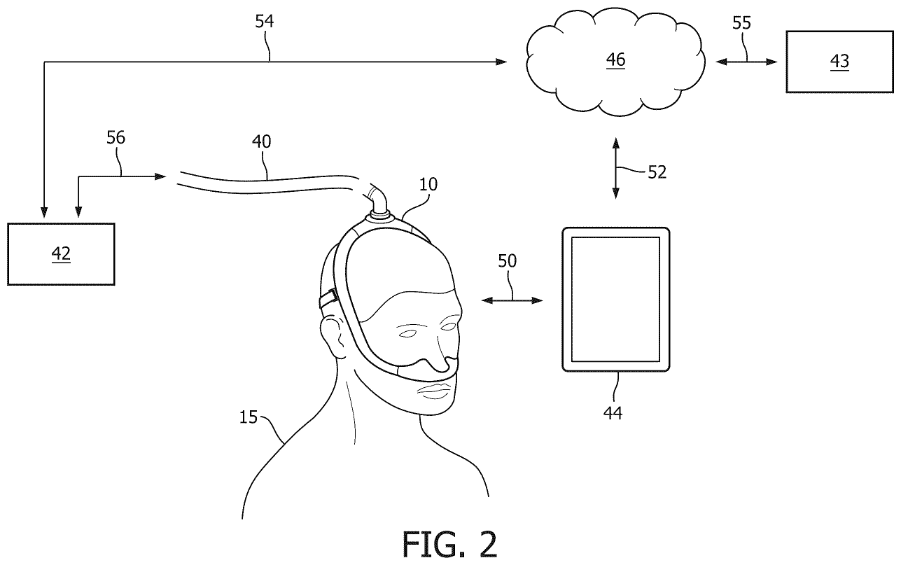

FIG. 2 indicates how the SMK 10 and its ensemble of sensors 12a, 12b monitor a patient 15 suffering from OSA during, e.g., sleep. A PAP device 42 connects to the SMK 10 through a hose 40, as indicated by arrow 56, and supplies positive pressure through the SMK 10 and to the patient to ameliorate the effects of OSA. It communicates with the cloud 46 in a bi-directional manner, as indicated by the arrow 54, to send parameters it measures (e.g., RR using an internal sensor, pressure values describing the air delivered to the patient, along with other patient information) and receive information related to PAP therapy, e.g., settings for pressure values, patient information. Simultaneously, the SMK 10 and the various sensors shown in FIG. 1A and 3A-3D and described above measure time-dependent waveforms, vital signs, and hemodynamic parameters from the patient. A Bluetooth® transmitter within the SMK 10 wireless transmits digitized versions of these signals, or derivatives calculated therefrom, to an external mobile device 44 (or alternatively a bedside hub), as indicated by arrow 50. The mobile device 44 can be a mobile phone, tablet computer, laptop computer, other computer, server, or wearable device; the bedside hub is typically a customized device, such as that shown in 10A-C.

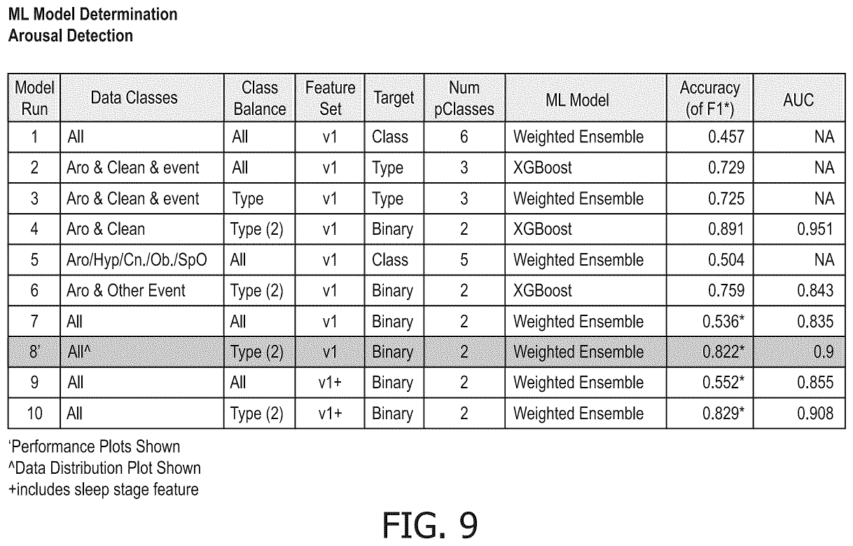

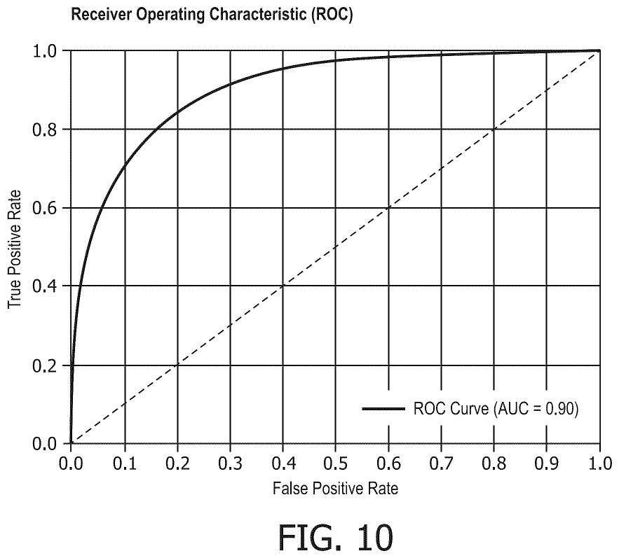

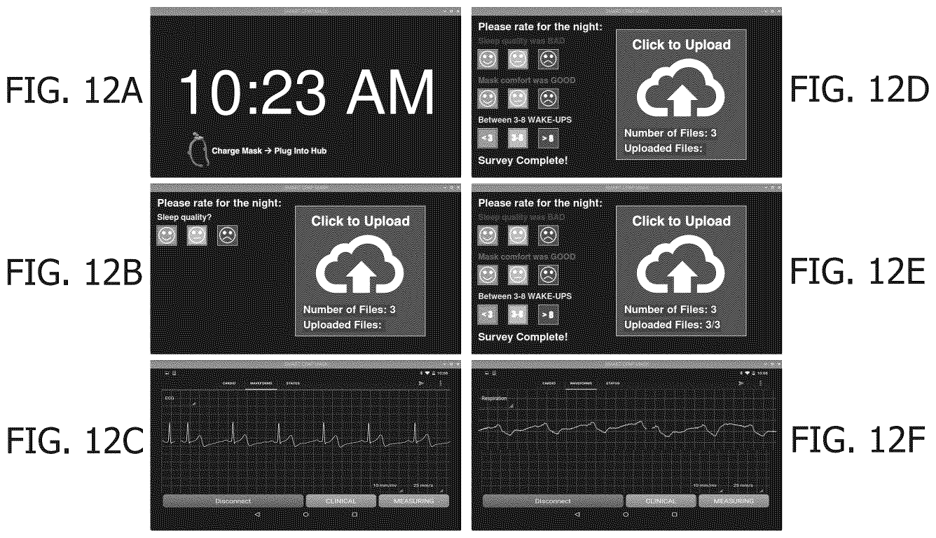

Once the mobile device 44 (or hub) receives information from the SMK 10, it transmits it to the cloud 46 as indicated by the arrow 52. The cloud 46 (e.g., Amazon Web Services, herein “AWS”) includes a collection of servers and software systems that can process SMK-measured waveforms, vital signs, and hemodynamic parameters to characterize the patient 15. Cloud-based systems like AWS include sophisticated algorithms and computational libraries, such as those based on ML and AI, to process data generated by the SMK 10 to produce reports and analyses, such as those shown in FIGS. 9 and 10. Such algorithms can be used to characterize the patient via RPM, or detect physiological events such as arousal and apnea (characterized, e.g., by low values of SpO2 and high values of HR) that may warrant a change in settings on the PAP device 42. Additionally, the mobile device 44 may include a user interface that poses survey-type questions to the patient 15, such as those shown in FIG. 12A, 12B, 12D, 12E that help engage the patient with their PAP therapy and supply information to the ML and AI algorithms to help improve characterization of the patient. In embodiments, for example, the user interface may pose surveys or questionnaires that help ascertain the patient's cognitive level after using the PAP device; this information, in turn, may be relayed back to the PAP device and used to improve the therapy it provides.

Third-party software systems 43, such as an electronic medical record (herein “EMR”) system, may also communicate with the cloud 46 in a bi-directional manner, as indicated by arrow 55. Here, the EMR may supply further information that may inform the ML and AI algorithms, such as the patient's medical history, medications they are taking, and results of blood tests, lab work, and other medical tests.

In embodiments, the PAP device 42 measures parameters that may complement those measured by the SMK. In embodiments, information measured by the PAP device 42 and sent to the cloud 46 (e.g., RR, respiratory tidal volumes, and the flow rate and pressure of air delivered to the patient), as indicated by arrow 54, may be combined and processed alongside of data collected by the SMK. For example, parameters such as respiratory tidal volumes may be incorporated in ML and AI models (such as those described in relation to FIGS. 9 and 10) to better estimate arousals. Or the RR measured by the PAP device 42 may be compared to that measured by the SMK to confirm the accuracy of this particular measurement.

In other embodiments, the system shown in FIG. 2 may operate in a closed-loop manner, wherein the SMK measures sleep-related parameters from the patient (e.g., number of arousals; physiological information such as HR, SpO2, BP, RR, SV, and CO), and in response sends a signal to the PAP device, which then adjusts a parameter related to the pressurized air delivered to the patient (e.g., its pressure and/or flow rate). Such a closed-loop system may lead to improved sleep and physiological outcomes for the patient.

In related embodiments, the SMK 10 may integrate with additional sensors that are positioned remotely from the actual mask but still measure complementary parameters. For example, referring to FIG. 3D, a multi-wavelength optical spectrometer 71 featuring a broadband light source 72 and specialized photodetector 70 can couple to the PAP hose 40 and measure gaseous compounds 74 (e.g., CO₂) that the patient exhales (as indicated by arrow 73) and propagate within the hose 40 during PAP therapy. The hose is an ideal location for the multi-wavelength optical spectrometer 71, as when attached to this component it can operate in a transmission-mode geometry; this (as compared to a reflection-mode geometry) typically improves the signal-to-noise ratio of the optical absorption spectra it measures. In addition to characterizing CO₂ in the patient's breathing—a parameter that, as described in the above-mentioned reference, may indicate BNP and thus decompensation of CHF patients—the multi-wavelength optical spectrometer 71 can measure optical spectra from the patient's exhaled breath that may indicate its composition.

The spectroscopic signals may then be processed with algorithms, e.g., those based on ML and/or AI, to characterize the rich molecular information contained in breath samples and thus provide insights on the patient's health. For example, exhaled human breath contains a wide range of VOC, VSCs and trace gases that can be indicative of various physiological and pathological processes occurring in the body. The presence of certain VOCs may indicate that the mask used in the SMK is aging, while VSCs may indicate the presence of bacteria. Spectroscopic techniques that may be performed with the multi-wavelength optical spectrometer 71 include optical absorption spectroscopy, infrared spectroscopy, Raman spectroscopy, laser spectroscopy, ultrafast laser spectroscopy, and optical comb filtering. The light source 72 for the spectrometer may be an LED, broadband light source (e.g., a tungsten light source), or a laser (e.g., a continuous-wave or pulsed laser, such as an ultrafast laser). Applications in health monitoring include disease diagnosis, as certain diseases and conditions can alter the composition of breath, leading to the presence of specific biomarkers than can be identified with optical spectroscopy. Specific examples include lung diseases, metabolic disorders, and gastrointestinal diseases. Additionally, by monitoring time-dependent changes in breath composition, optical spectroscopy can provide insights into disease progression, treatment efficacy, and may help assess the patient's response to therapies and guide personalized treatment approaches.

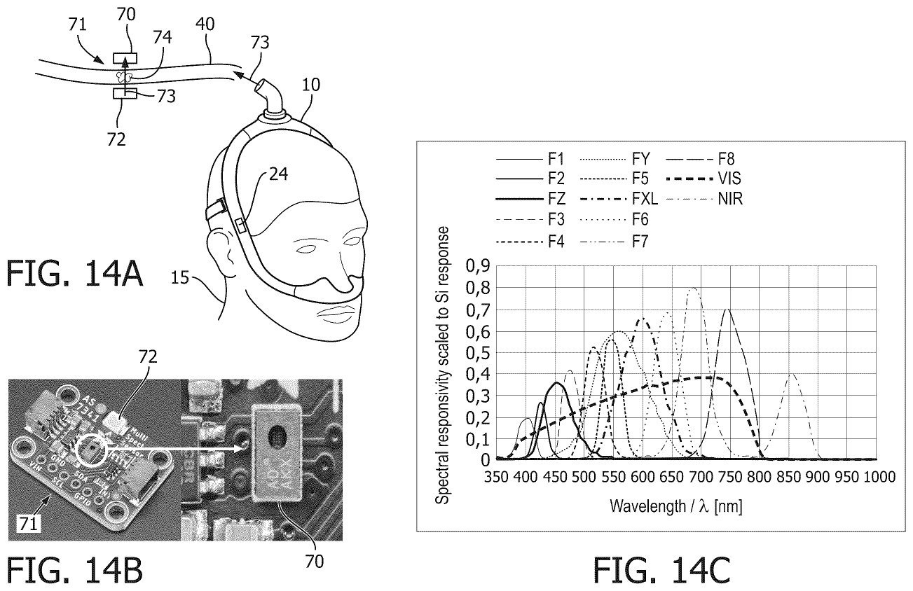

FIG. 14B and 14C, for example, show a multi-wavelength optical spectrometer 71 featuring the AMS AS7341 optical sensor functioning as the specialized photodetector 70. This component is a small-scale, chip-level system that works particularly well for the embodiment shown in FIG. 3D (and again in FIG. 14A), which requires the spectrometer to be coupled directly to the PAP hose 40, and thus be small and lightweight. The broadband light source 72 used in the spectrometer 71 is a white-light LED that emits optical radiation ranging from the infrared, through the visible frequencies, and into the ultraviolet. The AS7341 is a specialized sensor 70 that features a broadband photodetector covered by a set of optical filters based on computer-controlled micro-electromechanical systems (herein “MEMS”) that are controlled in software by setting certain programmable registers. FIG. 14C shows transmission spectra of the passbands of the optical filters, labeled in the figure as F1-F8 (corresponding to different narrowband optical filters in the visible spectral range with a passband of approximately Δλ=50 nm), FXL (corresponding to a relatively broadband filter ranging from λ=400-700 nm), VIS (λ=350-800 nm), and NIR (λ=800-900 nm). Other detectors similar to the AS7341 may be used to spectroscopically measure breath in other optical regions, e.g., the infrared.

During use, computer code running on the CPU within the SMK (described in more detail with reference to FIG. 4A) sets a specific register within the AS7341, which in turn activates a particular MEMS optical filter that transmits radiation characterized by a certain passband, as shown in FIG. 14C. The broadband photodetector disposed behind the MEMS optical filter detects the transmitted radiation and generates a photocurrent, and a 20-bit analog-to-digital converter coupled to the photodetector digitizes a voltage corresponding to the photocurrent measured across a known resistor. The digitized voltage represents a spectral data point corresponding to the central frequency of the optical passband. A single measurement typically takes a few milliseconds, and the process of setting a specific register and then measuring a corresponding signal is sequentially repeated until a complete optical spectrum is measured. Taken in combination, the small-scale multi-wavelength optical spectrometer 71 shown in FIG. 14A-C represents an ideal system for measuring different optical properties from breath exhaled from the patient 15 during the PAP therapy. Signals measures by this system, along with physiological information measured with the SMK 10, is transmitted to the cloud, where it is collectively analyzed to with ML and AI models to characterize the patient's health and improve their PAP therapy, as is described in more detail below.

In related embodiments, in place of the chip-level AS7341, the multi-wavelength optical spectrometer 71 can be based on more conventional technology, such as a broadband tungsten light source for irradiating the sample, a detection system featuring a diffraction grating or prism for dispersing optical radiation after it passes through the sample, a multi-pixel charge-coupled device (herein “CCD”) camera that detects the dispersed optical frequencies, and an external analog-to-digital converter and/or data-acquisition system that digitizes signals detected by each pixel in the CCD camera.

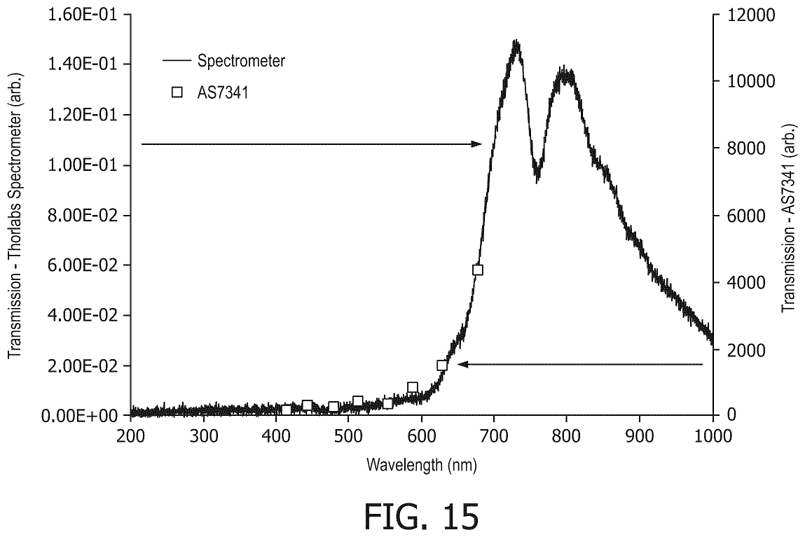

FIG. 15 shows frequency-dependent optical spectra taken from a human blood sample that is simultaneously measured at discrete frequencies with the AS7341 and a white-light LED (squares) and quasi-continuously with a conventional optical spectrometer manufactured by Thorlabs Inc. featuring a broadband tungsten light source and optical spectrometer featuring a diffraction grating and CCD camera (continuous dark line). The Thorlabs spectrometer provides quasi-continuous data points (e.g., one every nm) ranging from about 200-1000 nm, but is relatively large, heavy, and expensive compared to the AS7341; it is thus not well-suited for wearable applications like that leveraged for the SMK. In contrast, the optical spectra measured by the AS7341 is relatively limited in information, but each data point that the system measured in the range of λ=400-700 agrees well to that measured by the Thorlabs system. Importantly, and as described above, because of its size, weight, and cost, this chip-level system is well suited to integrate into the SMK.

### 2. Hardware Systems Used in the SMK

To effectively measure physiological information from the patient, the SMK includes a collection of sensing electronics positioned directly on the mask so that they contact portions of the patient's cheek and face, as shown in FIG. 1A, 3A, 3B, 3D and described in the accompanying text. These areas are uniquely suited for measuring physiological signals, mostly because they include dense capillary beds (ideal for measuring SpO2 and PR), are proximal to the mouth (ideal for measuring RR, sounds made during sleep, and compounds in the breath e.g., VOCs, VSCs, CO₂, etc.), and are proximal to large vessels in the chest and neck (for measuring hemodynamic parameters such as SV, CO, and FLUID). The sensing electronics are typically stand-alone digital systems that measure a specific analog signal from the patient, digitize the signal on-board with an analog-to-digital converter, and then transmit the signal through a wired serial bus, as described in more detail below. This approach has advantages to transmitting weak analog signals over long, ‘lossy’ cables and then digitizing them. Each stand-alone digital system requires power (typically 1.8-5.0V) provided by a single-board computing platform within the control system and its accompanying Li:ion battery. Typically, the Li:ion battery generates a voltage that ranges from 4.2V (fully charged) to 3.6V (depleted); voltage regulators on the single-board computing platform convert this into the voltage required by the sensing electronics.

The single-board computing platform within the SMK's control system controls the sensing electronics by operating them through a series of digital commands, and processing the signals they generate. It features a CPU for controlling the system and running algorithms that process the sensor-measured data, Flash and RAM memory, a collection of AFEs for controlling the sensors and processing the signals they generate, and one or more wireless transmitters (e.g., Bluetooth®, Wi-Fi, cellular) for sending digitized vital signs, hemodynamic parameters, and time-dependent waveforms to a remote mobile device and/or hub. To control the sensing electronics distributed around the SMK, the single-board computing platform connects to the flexible ribbon cables, which plug into its left and right-hand sides using separate multi-pin connectors. Collectively, the multi-pin connectors and flexible ribbon cables supply power and ground to the sensing electronics, and communicate with them over a serial bus that operates digital protocols such as an inter-integrated circuit protocol (herein “I2C”), inter-integrated circuit sound (herein “I2S), serial peripheral interface (herein “SPI”), universal asynchronous receiver/transmitter (herein “UART”), or comparable protocols. The various protocols—I2C, 12S, SPI, UART—typically require serial lines (i.e. conductive cables in the flexible ribbon cable, as shown in FIG. 3E) for a clock, data, and chip-select. Because the communication protocols operate on a bus, they can simultaneously communicate with multiple sensing electronics by using the chip-select line, which identifies a specific sensor for communication. Typically, the CPU includes multiple busses for communication. In a preferred embodiment, the CPU is the STM32u545/575/585 component manufactured by ST Microelectronics.

The single-board computing platform within the SMK's control system may also directly integrate with 3^{rd}-party systems, such as a website on the Internet (e.g., social media platforms, or AI-based system such as ChatGPT), Amazon's Alexa, or ‘smart’ networks within the patient's home. In embodiments, for example, the single-board computing platform may include a 2-way speech-to-text converting system that allows the patient to verbally send commands to the SMK to adjust a particular setting, and the SMK to audibly convey to the patient parameters that it measured during the previous night's sleeps, e.g., arousals and/or physiological information.

Referring to FIG. 4A and 4B, in embodiments the control system in the SMK includes a single-board computing platform 14 that includes the following components (note that not every component in the PCB is described below):

| Component | | |
|---|---|---|
| Number | Component | Function |
| 102a | Multi-pin connector | Connects CPU in single-board |
| | to flexible ribbon | computing platform to sensors |
| | cables (left side) | distributed in SMK - provides power, |
| | | ground, and serial connections |
| 102b | Multi-pin connector | Connects CPU in single-board |
| | to flexible ribbon | computing platform to sensors |
| | cables (right side) | distributed in SMK - provides power, |
| | | ground, and serial connections |
| 104 | Bluetooth ® | Wirelessly transmits numerical and |
| | module | waveform data to external mobile |
| | | device, external hub, etc. |
| 106 | USB-C connector | Wired connection to charge Li:ion |
| | | battery, transfer numerical and |
| | | waveform data to external mobile |
| | | device, external hub, etc. |
| 108 | CPU | Microprocessor with internal memory |
| | | and processing power for running |
| | | real-time operating system and |
| | | algorithms, multiple buses (e.g., I2C, |
| | | I2S, SPI, UART) for communicating |
| | | with peripheral devices |
| 110 | MAX86176 IC | AFE for controlling ECG, EMG, and |
| | | optical (PR, SpO2) measurements |
| 111 | 3-axis | Determines motion along x, y, and |
| | accelerometer | z-axes; these data are used to |
| | | determine patient motion and posture |
| 114 | Fuel gauge | AFE for managing power and voltage |
| | | levels from Li:ion battery |
| 118 | On/off button | Button for turning the platform on |
| | | and off |
| 120 | Connector | Connector for Li:ion battery |
| 122 | Connector | Extra component |
| 124 | Flash memory | 32 GB of non-volatile memory for |
| | | storing information collected during |
| | | the PAP therapy |

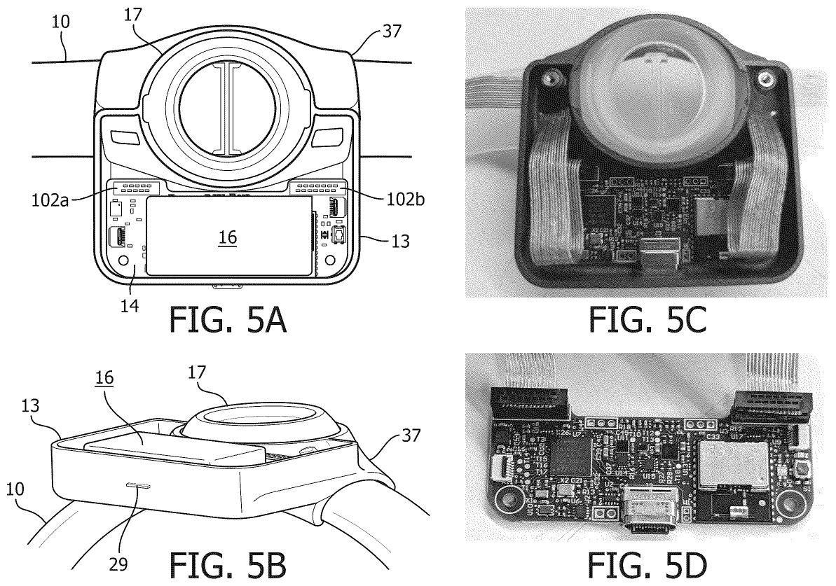

FIG. 5A and 5B show the single-board computing platform 14 integrated into a mechanical housing 13 posited at a top portion 37 of the SMK 10. The mechanical housing 13 includes openings for the multi-pin connectors 102a, 102b disposed on both the left and right-hand side of the single-board computing platform 14; these connect to flexible ribbon cables (not shown in the figure, but shown in FIG. 3E, 5C, and 5D) that attach to tubes in the PAP mask, support and power the patient-contacting sensing electronics, and provide 2-way serial interfaces between the sensing electronics and the CPU within the single-board computing platform 14. The enclosure 13 houses a Li:ion battery 16 that powers the system, and additionally includes an opening 29 for a USB-C cable. Plugging the cable into the opening 29 charges the Li:ion battery 16 and also downloads data collected during the patient's sleep, and stored in the single-board computing platform's internal Flash memory, into an external device (e.g., the hub and/or mobile device).

FIG. 5C and 5D show photographs of, respectively, the PCB within the enclosure, and the PCB outside of the enclosure. In both cases, flat, flexible ribbon cables attach to the multi-pin connectors on both sides of the PCB, and attach the PCB to the various sensors distributed in the SMK, as shown by the photograph in FIG. 3E.

The top portion 37 of the mechanical housing includes a ‘swivel joint’ 17 that an elbow-shaped connector (not shown in the figure) on a distal end of the PAP hose connects to. The hose connects to the PAP device and supplies positively pressured air to the SMK 10. An important design consideration of the enclosure 13 is to not impede rotation of the elbow-shaped connector within the swivel joint 17, as this rotation is important to keep the SMK 10 comfortably connected to the patient even while they toss and turn, and generally move, during sleep.

In embodiments, the dimensions of the enclosure are as follows: 55 mm×30 mm×10 mm. The enclosure, combined with the PCB and Li: ion battery housed therein, is between 15-20 grams. Sensors connected to the enclosure through the ribbon cables and attached to the PAP mask weigh an additional 5-10 grams.

### 3. Clinical Studies

The SMK, with its sensing electronics and single-board computing platform, collects time-dependent waveforms from the patient's face and cheek during sleep. The CPU within the platform then processes these waveforms with algorithms, e.g., those that digitally filter the waveforms, transform them (e.g., using Fourier, Laplace, or similar transforms), and/or perform beatpicking operations to detect fiducial markers that characterize, e.g., heartbeat-induced and respiratory pulses. These can be, for example, a foot of a pulse, amplitude of a pulse, area of a pulse, pulse-to-pulse separation, variation in this separation, etc. Algorithms running on the CPU can then process these fiducial markers to determine vital signs and hemodynamic parameters, e.g., HR, HRV, PR, RR, SpO2, SV, CO, and FLUIDS corresponding to the patient. Such algorithms are described in the prior art. For example, U.S. Pat. No. 11,357,453 to Banet et. al describes many of them, and is incorporated herein by reference.

In alternate embodiments, the SMK and its associated systems simply collect time-dependent waveforms from the patient, and then forward these (using wired or wireless means) to an external system such as the bedside hub, which then processes them as described above. Stated another way, in this embodiment, the SMK is simply a data collector/router that supplies raw data to the external system for more sophisticated analysis. Such a configuration has certain advantages, as it offloads much of the signal processing reduces the computing cycles of the SMK's CPU, thereby conserving battery life and reducing the general requirements of the CPU (e.g., its size and cost). Additionally, this can mean a smaller (and lighter) Li:ion battery powers the system, thereby increasing patient comfort.

More specifically, in embodiments, the SMK described above determines vital signs (e.g., HR, RR, SpO2) and hemodynamic parameters (e.g., SV, CO, FLUIDS) by collectively measuring and processing time-dependent ECG, PPG, IPG, pressure, EMG, and ACC waveforms, as shown in FIG. 6A-F (note: BR and IPG waveforms have a similar morphology, and thus for simplicity only IPG waveforms are shown in FIG. 6C). Analog-to-digital converters within the SMK digitize the waveforms shown in the figure, which are originally measured in analog form, at 250 Hz. ECG, PPG, and IPG waveforms shown, respectively, in FIG. 6A, 6B, and 6C, typically include a heartbeat-induced ‘pulse’; these are indicated in the figure by dashed lines 140a, 140b (ECG waveform), 141a (PPG waveform), and 141b (IPG waveform). The temporal separation of the pulses in the ECG waveform, as indicated by dashed lines 140a and 140b, is inversely related to HR, as indicated in FIG. 6A; this value is typically between 30-200 beats-per-minute (herein “bpm”). The SMK can also measure HR from pulse-to-pulse separation in both the IPG waveform and PPG waveform. For example, dashed lines 141a and 141b indicate, respectively, the foot of pulses within these waveforms; separation of this feature in neighboring pulses indicates HR. The foot is often chosen as such a fiducial as it indicates when a pulsatile bolus of blood arrives at the capillary beds underneath the optical sensor (in the case of PPG waveforms), or the relatively large arteries proximal to sense and drive electrodes (in the case of IPG waveforms).

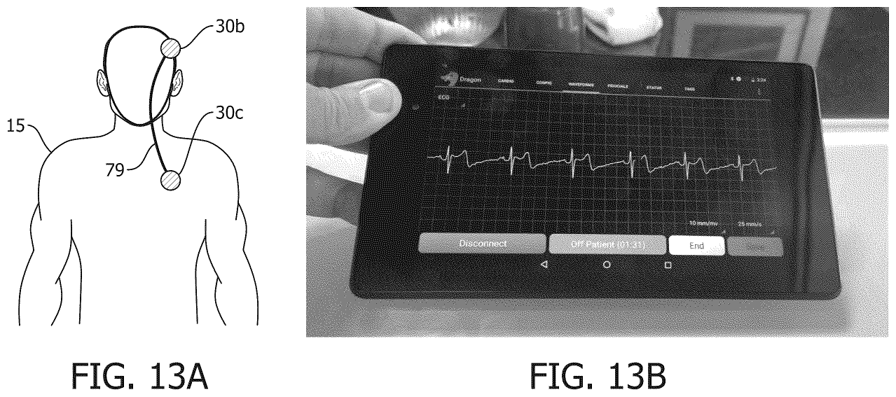

FIG. 6A shows an ECG waveform measured by the SMK when used in the configuration shown in FIG. 13A, described in more detail below. The ECG waveform includes a heartbeat-induced ‘QRS complex’, i.e. a sharp time-dependent spike that informally marks the beginning of each cardiac cycle. Compared to other physiological waveforms, ECG waveforms typically have relatively good signal-to-noise ratios and are easy to analyze with beat-picking algorithms; they are thus often used to measure HR, and QRS complexes function as fiducial makers for analyzing some of the more complex waveforms described below. FIG. 6B shows a PPG waveform, which is measured by one or both optical sensors deployed in the SMK, and indicates volumetric changes in underlying capillaries caused by heartbeat-induced blood flow. As is well known in the art, the AC and DC components of PPG waveforms measured with optical radiation in the red (λ˜660 nm) and infrared (2 ˜940 nm) can be collectively processed to determine values of SpO2.

The IPG waveform shown in FIG. 6C also includes both AC and DC components: the DC component indicates the amount of fluid in the face and neck region by measuring baseline electrical impedance (i.e. FLUIDS); the AC component, which is shown in FIG. 6C, tracks blood flow in the face and neck, and represents the pulsatile components of the IPG waveform. The time-dependent derivative of the AC component includes a well-defined peak that indicates the maximum acceleration of blood flow in the thoracic vasculature. Both the AC and DC components can be processed along with a parameter called left ventricular ejection time (herein “LVET”) and an equation referred to above (e.g., the Sramek-Bernstein or Kubicek equations, or an equivalent thereto) to determine SV. LVET, a parameter included in both the Sramek-Bernstein and Kubicek equations, indicates the temporal separation between the opening and closing of the aortic valves.

More specifically, during an IPG measurement, the sense electrodes measure a time-dependent voltage (V) that varies with resistance (R) encountered by the injected current (I). This relationship is based on Ohm's Law, shown below:

$$V = I \times R$$

The impedance circuit (e.g., the impedance AFE) measures the voltage as described above, and digitizes it with an internal analog-to-digital converter. The microprocessor receives the digitized voltage, along with other parameters described below, and using computer code processes it with an equation (e.g., the Sramek-Bernstein equation or Kubicek equation, or a mathematical variation thereof) to calculate SV. These equations, which are based primarily on a model that assumes volumetric expansion, are shown below:

### Sramek-Bernstein Equation

$$S V = δ \frac{{L}^{3}}{4.25} \times \frac{{( \frac{d Z ( t )}{d t} )}_{max}}{Z 0} \times LVET$$

### Kubicek Equation

$$S V = ρ \frac{{L}^{2}}{Z {0}^{2}} \times {( \frac{d Z ( t )}{d t} )}_{max} \times LVET$$

where Z(t) represents the IPG waveform (i.e. the AC component of the waveform), δ represents compensation for body mass index (\sqrt{BMI}/24 kgm²), Z0 is the base impedance (i.e. the DC component of the waveform), L is estimated from the distance separating the sense and drive electrodes, ρ is the static resistance of blood (135 Ωcm), and LVET is the time separating the opening and closing of the aortic valve. LVET and can be determined directly from the IPG waveform by analyzing a feature called a ‘dichrotic notch’, which is typically present in each heartbeat-induced in the waveform, or from an HR value using an equation called ‘Weissler's Regression’, shown below:

$$LVET = - 0.0017 \times H R \times 0.413$$

When used in Weissler's Regression, HR can be determined from a number of different signals, e.g., the ECG, PPG, or IPG waveform.

This equation and several mathematical derivatives are described in detail in the following reference, the contents of which are incorporated herein by reference: Bernstein et. al, ‘Impedance Cardiography, Pulsatile blood flow and the biophysical and electrodynamic basis for the stroke volume equations’, Journal of Electrical Bioimpedance, Vol. 1, p. 2-17, 2010. Both the Sramek-Bernstein Equation and the Kubicek Equation, assume that (dZ(t)/dt)ₘₐₓ/Z0 represents a radial velocity (with units of Ω/s) of blood due to volume expansion of the aorta.

In the equations above, the parameter Z0 will vary with fluid levels. Typically, a high resistance (e.g., one above about 30Ω) indicates a dry, dehydrated state. Here, the lack of conducting thoracic fluids increases resistivity in the patient's chest. Conversely, a low resistance (e.g., one below about 19Ω) indicates the patient has more thoracic fluids, and is possibly overhydrated. In this case the abundance of conducting thoracic fluids decreases resistivity in the patient's chest. The impedance circuit and specific electrodes used for a measurement may affect these values. Thus, the values can be more refined by conducting a clinical study (e.g., one with a large number of subjects, preferably with a large variability in their fluid status), and then empirically determining ‘high’ and ‘low’ resistance values.

FIG. 6D is the output of the pressure sensor, and represents a direct measurement of RR, as described in detail above. In addition to providing accurate RR values, this waveform can be used to filter out respiration artifacts in other waveforms, such as the PPG and IPG. For example, RR measured from the pressure waveform may be incorporated into a digital filter called an adaptive filter. The adaptive filter then sets its passband to exclude physiological events that specifically occur at the RR frequency. In embodiments, the filtering described by the passband is done using an infinite impulse response (herein “IIR”) digital filter, as is known in the art.

FIG. 6E shows an EMG waveform featuring three unique pulses, shown by dashed lines 147a, 147b, and 147c, and each indicating electrical activity caused by muscle movement in the patient's face (in this case, simulated by jaw clenching around 170, 177, and 181 seconds). The pulses are comparable to natural motions that the patient undergoes while sleeping and, for example, experiencing an arousal. To determine this waveform, the sense electrodes used to measure the IPG waveform in FIG. 6C simultaneously sense bio-electric signals generated by electrical activity associated with the muscle movement.

The sense electrodes connect to both the IPG AFE (MAX30009) and the ECG AFE (MAX86176), the latter serving as a proxy to an EMG circuit. The IPG AFE filters out the bio-electric signals, but they pass through filters within the ECG AFE; an internal differential amplifier then electronically calculate their difference and gains up the resulting value to yield the waveform shown in FIG. 6E. Notably, bio-electric signals generated by the muscle noise are quite different on one side of the face compared to the other, and thus the pulses indicated by dashed lines 147a, 147b, and 147c feature relatively good signal-to-noise ratios. In contrast, heartbeat-induced bio-electric signals measured at different sides of the face by the sense electrodes show very little difference, and thus ECG waveforms cannot typically be measured from this region. However, as indicated by FIG. 13A and 13B, the ECG AFE can measure ECG waveforms with relatively good signal-to-noise ratios using one electrode within the SMK that contacts one side of the patient's face, and a second ‘satellite’ electrode that adheres to the patient's chest.

FIG. 6F shows an ACC waveform, measured by the accelerometer within the SMK. As indicated by FIG. 4A and described above, the accelerometer is typically located directly on the PCB, which in turn is disposed near the top of the patient's head. The accelerometer measures ACC waveforms similar to that shown in FIG. 6F along 3 unique axes (x, y, and z), and typically also includes a gyroscope to measure the patient's angular movements; algorithms can process each of these to estimate movements that may, in turn, indicate arousal during sleep. Other algorithms determine a vector magnitude of the patient's motion by squaring values of ACC waveforms measured along the x, y, and z-axes, and then taking the square root of the resultant value. Once measured, the vector magnitude can indicate the degree of the patient's motion. Algorithms known in the art can process the individual ACC waveforms measured along the x, y, and z-axes to determine the patient's posture. This value can be important during sleep, as it may indicate sleeping postures that are more conducive to apnea events, e.g., when the patient is sleeping on their back, as well as those that are less conducive, e.g., when the patient is sleeping on their side or stomach.

In related embodiments, the microprocessor within the PCB may deploy signal processing techniques in addition to IIR digital filters to improve signal-to-noise ratios of the waveforms and heartbeat-induced pulses shown in FIG. 6A-6F. These include smoothing, averaging, beatstacking, and other types of digital filtering.

Parameters related to SYS, DIA, and MAP BP can be determined by analyzing the time difference between pulsatile features in different waveforms. For example, algorithms operating in firmware on the SMK can calculate time intervals between the QRS complex and fiducial markers on each of the other waveforms. One such interval is the time separating the foot of the pulse in the IPG waveform (FIG. 6C) and the foot of the PPG waveform (FIG. 6B). This is shown, for example, by a first dashed line 141a indicating the foot of a pulse in the PPG waveform (FIG. 6B), and a second dashed line 141b indicating the foot of a pulse in the IPG waveform (FIG. 6C). This time interval typically indicates PTT and/or VTT, and is indicated in the figure; it is typically between 10-100 ms, and is inversely related to BP. Additionally, the dashed line 144a indicates the QRS complex of a pulse in the ECG waveform (FIG. 6A), and the temporal separation between this fiducial and the foot of the pulse in the PPG waveform indicated by dashed line 141a is the PAT. Alternatively, the PAT can be determined from the ECG QRS indicated by dashed line 144a and the foot of the pulse in the IPG waveform as indicated by dashed line 141b. Typically, PAT values are slightly longer than PTT/VTT values, usually in the range of 50-200 ms.

In embodiments, fiducials corresponding to the feet of pulses can be interchanged with those indicating a peak of a pulse, e.g., a peak of a pulse in the IPG waveform (FIG. 6C) or PPG waveform (FIG. 6B). In general, any set of time-dependent fiducials determined from waveforms other than ECG can be used to determine PTT and VTT. Collectively, PAT, PTT, VTT, and other time-dependent systolic time intervals extracted from pulses in the four physiologic waveforms described above are inversely related to BP.

Typically, BP-measurement methods based on systolic time intervals indicate changes in BP; they require calibration from a cuff-based system (e.g., manual auscultation or automated oscillometry) to determine absolute values of BP. Typically, such calibration methods provide initial BP values and patient-specific relationships between BP and PAT, PTT, and VTT. During a cuffless measurement, these values are measured in a quasi-continuous manner, and then combined with the values of BP determined during calibration to yield quasi-continuous values of BP. Such calibrations typically involve measuring the patient multiple (e.g., 2-4) times with a cuff-based BP monitor employing oscillometry, while simultaneously collecting PAT, PTT, and VTT values like those described above. Each cuff-based measurement results in separate BP values. Calibrations typically last about 1 day before they need to be repeated.

Accurate determination of BP values from PAT, PTT, and VTT may also require determination of a patient-specific constant that relates changes in these transit times to changes in BP. Such a patient-specific constant can be determined by measuring the transit times and calibration measurements at different BP values, and then determining them through linear interpolation. Alternatively, the constants can be estimated from meta data and population models, or by analyzing the shape of pulses in the time-dependent waveforms. In other embodiments, the constants are determined from large data sets using ML and AI.

In related embodiments, one of the cuff-based BP measurements is coincident with a ‘challenge event’ that alters the patient's BP, e.g., squeezing a handgrip, changing posture, or raising their legs. This imparts variation in the calibration measurements, thereby improving sensitivity of the post-calibration measurements to BP swings. In other embodiments, a ‘universal calibration’ (e.g., a single calibration for all patients) can be used for the BP measurement. In other embodiments, the BP measurement is left uncalibrated, and only relative measurements of BP are calculated.

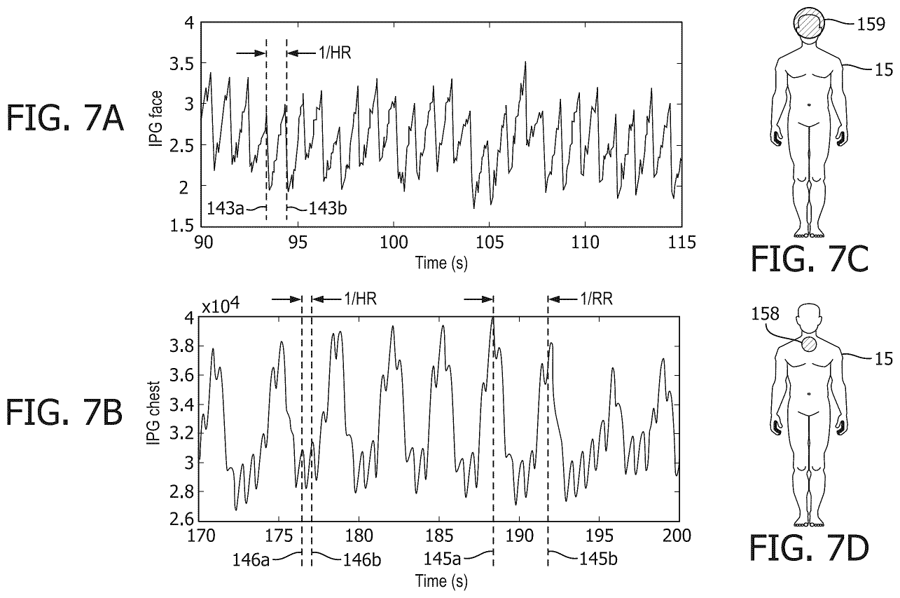

Referring to FIG. 7A-7D, IPG waveforms measured from the face with the SMK include several features that makes them particularly well suited for measuring transit times (e.g., PAT, PTT, VTT) and physiological parameters (e.g., BP, SV, and CO). For example, and somewhat surprisingly, pulses in these waveforms show very little modulation due to respiration. The IPG waveform in FIG. 7A is measured from the face of a patient 15 with the SMK positioned on the patient's head, as indicated by the circle 159 shown in FIG. 7C. Here, sense and drive electrodes used in the IPG measurement are distributed in the SMK as indicated by FIG. 1A and 3B. These electrodes generate time-dependent waveforms with well-defined pulsatile features, as indicated by dashed lines 143a, 143b in FIG. 7A; HR is calculated from these, as is indicated in the figure. The rise time of these pulsatile figures is extremely sharp, indicating a rapid acceleration of blood flow to the face with each heartbeat. Importantly, the IPG features very little modulation due to respiration.

In contrast, FIG. 7B and 7D show, respectively, an IPG waveform measured from the chest of a patient 15, as indicated by the circle 158. Here, the IPG waveform shows both heartbeat-induced features as indicated by dashed lines 146a, 146b, and respiration-induced features as indicated by the dashed lines 145a, 145b; the separation between these pairs of dashed lines indicates, respectively, HR and RR. The IPG waveform in FIG. 7B shows well-defined modulation due to respiration because breathing changes the capacitance—and hence impedance—in the patient's chest, and most notably in the underlying lungs. Respiration-induced modulation of the IPG waveform dominates the signal, obscuring to some extent the heartbeat-induced features and making it difficult (or in some cases impossible) to extract and analyze them from the signal. Such a physiological change is not present in the patient's face, and thus IPG waveforms measured from this region typically lack a respiration component, as indicated by FIG. 7A. This makes such signals relatively easier to extract and analyze, ultimately leading to improved accuracy for measuring parameters that utilize them (e.g., HR, BP, SV, CO).

When compared to FIG. 7B, the relatively rapid rise of heartbeat-induced pulses in the IPG waveform in FIG. 7A may be due to blood flowing rapidly into the face from the common carotid arteries, located on each side of the neck. These arteries branch into smaller vessels, called facial arteries, as they reach the face. The facial arteries then divide further into numerous smaller arteries, delivering oxygenated blood to different regions of the face. This vasculature is in contrast to that in the chest, which features the large and relatively supple aorta. IPG signals originating from the aorta include features from volumetric expansion of this vessel (which is the largest artery in the human body), along with blood flow-induced alignment of cigar-shaped red blood cells called erythrocytes; both of these physiological events increase electrical conduction in the chest and thus decrease impedance measured therefrom. However, their relative contribution to the IPG signal typically varies on a patient-by-patient basis, and can thus be difficult to tease out and incorporate into a mathematical model, such as the Sramek-Bernstein or Kubicek equation. Moreover, fluids in the chest are somewhat common, particularly for patients suffering from CHF, and these fluids can artificially increase baseline values of the IPG waveform; this, in turn, can artificially increase a parameter (Z0) in the above-mentioned equations, thereby decreasing their accuracy.

In contrast, blood vessels in the face and neck are less flexible than the aorta and typically surrounded by muscle. Thus, IPG signals emanating from them are driven mostly by alignment of the erythrocytes, as opposed to volumetric expansion, making it relatively easier to develop physiological models that describe them. Such models are similar to those used to accurately calculate SV from IPG signals measured from the brachial arteries, as described by an impedance-based measurement called trans-brachial electrovelocimetry (herein “TBEV”). U.S. Pat. No. 10,278,599 to Banet et al. describes the TBEV measurement, and is incorporated herein by reference. Additionally, conditions like CHF increase fluids in the chest but not necessarily the neck, meaning the inaccuracies they cause SV-calculating equations may impact chest-worn sensors but not the SMK.

The sharp rise of pulses in IPG waveforms measured from the face also yields a relatively accurate determination of the foot of these pulses. This, in turn, reduces error in calculating transit times such as PAT, PTT, and VTT, ultimately determining the accuracy to which BP is calculated from these signals.

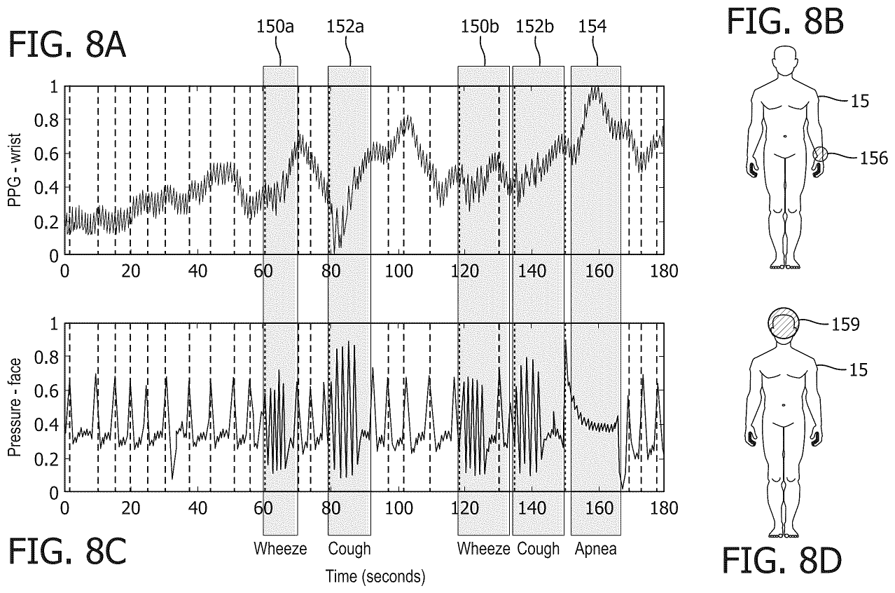

The SMK measures pressure waveforms that are particularly effective at indicating respiratory events; processing them can yield accurate values of RR, as well as other respiratory events, such as coughing, wheezing, and apnea. FIG. 8A-8D indicates this through a comparison to PPG waveforms measured from the wrist and hands using optical sensors, e.g., those within conventional pulse oximeters, fitness trackers, ‘smart’ watches, and ‘smart’ rings. Here, as indicated by FIG. 8A and 8B, the patient 15 wears a smart watch containing an optical sensor that contacts their wrist, as indicated by a first circle 156. At the same time, as indicated by FIG. 8C and 8D, the patient 15 wears an SMK with a pressure sensor near their mouth, as indicated by a second circle 159. Over a period of about 3 minutes, the optical sensor measures a PPG waveform (usingλ=940 nm) from the patient's wrist in a reflection-mode geometry, and the pressure sensor measures a pressure waveform, similar to that shown in FIG. 6D, from the patient's mouth. During the measurement period, the patient 15 intentionally initiates a simulated wheezing breathing pattern around 60s and 120s, as indicated by boxes 150a, 150b. The patient 15 also simulates coughing around 80s and 135s, as indicated by boxes 152a, 152b, and an apnea even where they hold their breath for about 30s around 150s. The optical and pressure sensors measure, respectively, PPG and pressure waveforms continuously (with a sampling rate of 250 Hz) throughout the 3-minute measurement period, including during the wheezing, coughing, and apnea ‘challenges’.

FIGS. 8A and 8C show, respectively, time-dependent graphs of the PPG and pressure waveforms. The gray dashed lines that span both figures indicate annotated respiration events. Referring first to FIG. 8A, the PPG waveform shows quasi-periodic, well-defined pulses throughout the measurement period, with each pulse corresponding to an individual heartbeat of the patient. There is some modulation of an ‘envelope’ defining peaks and nulls of the pulses, but this modulation does not appear to specifically correspond to respiration events, as indicated by the dashed gray lines. The PPG waveform shows some variability during periods of wheezing, cough, and apnea, as indicated respectfully by boxes 150a 150b, 152a, 152b, and 154, although this variability does not appear to include specific features that indicate these particular challenges, i.e. the variability shown in box 150a corresponding to wheezing does not appear distinguishable to that shown in box 152a for coughing, or box 154 for apnea.

In contrast, and referring to FIG. 8C, the pressure waveform appears superior in indicating respiration events and challenges due to wheezing, coughing, and apnea. During each breath indicated by the gray dashed lines, for example, the pressure waveform shows a well-defined pulse corresponding exactly to the respiratory event. During apnea, as indicated by box 154, the respiratory-induced pulses completely disappear. Periods of wheezing indicated by boxes 150a, 150b show pulses with significantly higher amplitude than the heartbeat-induced pulses, and more similar in amplitude to respiratory pulses; however, they occur at relatively high frequencies (e.g., >2 pulses/second) for a short ‘burst’ that corresponds to the wheezing event. Coughing as indicated by boxes 152a, 152b yields pulses with even higher amplitudes than those corresponding to wheezing. They occur at a frequency corresponding to the cough frequency (about 1 pulse every 1-2 seconds) that is typically lower than the wheezing frequency, and have a distinct shape that differs from the pulse generated by wheezing.

The PPG and pressure waveforms shown, respectively, in FIG. 8A and 8C, indicate that pressure waveforms measured directly from the patient's mouth are generally superior to PPG waveforms measured near the wrist and fingers for detecting respiratory events, such as normal breathing, wheezing, coughing, and apnea. PPG waveforms remain valuable for determining SpO2 and PR which remain important parameters for characterizing OSA and the efficacy of the PAP therapy.

### 4. ML Models for Processing Data Generated by the SMK

In embodiments, cloud-based ML models can process physiological information (e.g., numerical values of vital signs and time-dependent waveforms) generated by the SMK to characterize a patient. Such ML models were first generated using data measured during polysomnography from patients undergoing lab-based sleep studies used to screen them for OSA. These data were categorized in the Sleep Heart Health Study Database (herein “SHHSD”), and were similar (but not identical) to that measured by the SMK. During the sleep studies, clinicians having expertise in sleep disorders annotated the following sleep-related conditions (referred to in the SHHSD as ‘Data Classes’): arousals, hypopnea, central apnea, obstructive apnea, and SpO2 desaturation events. Each annotated Data Class was documented by the clinicians, meaning the event was observed and its severity and temporal occurrence was noted and recorded. Additionally, during time periods where no Data Class was present, the data was labeled as ‘Clean’.

To generate the reports shown in the figures, a series of ML-based models were first tested on a first cohort of patients in the SHHSD using the annotated Data Classes (and particularly arousal) and the associated physiological information leading up to them. Once the models were optimized, they were tested on a second cohort of patients and used to predict the Data Classes. And because these events were previously annotated by the sleep clinicians and noted in the SHHSD, this approach made it possible to test parameters such as accuracy, specificity, and selectivity of various models for predicting Data Classes based on physiological information measured with the SMK. Once this was done the ideal model was selected.

For the various ML models, ‘fiducials’ as described above, along with calculated parameters in the SHHSD, were detected from time-dependent waveforms collected using 30-second windows surrounding the different Data Classes. This information—which was chosen because of its similarity to data measured by the SMK—included: 1) HR and associated variability metrics (e.g., standard deviation, root-mean-squared standard deviation, mean HR variability); 2) RR and associated variability metrics (e.g., instantaneous RR, breath-to-breath time intervals, and additional statistical measures such as standard deviation, mean, max, min values); 3) SpO2 and associated variability metrics (e.g., variability, mean, max, min); and 4) motion-related information extrapolated from the EMG measurement (this was used as a surrogate for ACC waveforms, as no accelerometer data was present in the SHHSD). Data were then split in multiple ways to evaluate the efficacy of different classification models (binary vs. multiclass) and to understand the impact of Class imbalance on the dataset, and then balanced based on: 1) individual Data Class; 2) grouping of Data Classes, e.g., arousals, clean data, and other events; or 3) binary, e.g., arousals vs clean data; arousals vs. other events, and arousals vs. everything else.

Once these parameters were extracted from the data set, they were formatted and processed with various ML models available through AWS. In embodiments, other cloud-based platforms, e.g., Microsoft's Azure or any other comparable cloud-based software system, may be used in place of AWS. In other embodiments, the cloud-based software system is integrated directly with a regenerative AI model, such as that used with a system such as ChatGPT.

Performance of the ML model was evaluated based on Accuracy (or alternatively F1 score, defined below) and the area under the curve (herein “AUC”) calculated from receiver operating characteristic (herein “ROC”) plots like that shown in FIG. 10. ROC plots show the dependence of true positive rate (y-axis) on the false positive rate (x-axis). The AUC calculated from them provides an aggregate measure of performance across all classification types, where 1 indicates a perfect classifier, a value of 0.9-1.0 considered excellent, 0.8-0.9 very good, 0.7-0.8 good, 0.6-0.7 satisfactory, 0.5-0.6 unsatisfactory, and 0.5 or lower a completely random classifier.

FIG. 9 summarizes the results for detecting Data Classes using the above-described ML models. Specifically, the figures describe different models tested, the Data Classes used, how these data were processed, the specific ML model used, the resulting accuracy (or F1 score for ensemble averages), and AUC. Data Classes, in this case, refer to the annotated polysomnography events in the SHHSD, and include EEG-based arousal, respiratory events, oximeter artifacts, arousals, central apnea, obstructive apnea, and hypopnea. These annotations are based on assessment by trained and certified technicians. A Data Class is considered ‘Clean’ if none of these events occur.

In FIG. 9, the term ‘Class Balance’ refers to the distribution of each of the Data Classes within the dataset. This is important to ensure that the data are not biased by having too many or too few of the different Data Classes. For example, if only 5% of the data is composed of annotated arousals, the ML Model would skew toward false negatives. Likewise, if the dataset included a disproportionate number of annotated hypopnea (or any other) events, this too would skew the ML model's performance towards these particular events. As such, during the analysis, the Data Classes are ‘balanced’ such that the target Data Class (in this case arousals) is approximately 50% of the data, and that the other Data Classes are represented in approximately the same proportions expected in the population. In the table ‘All’ means that the data is split evenly between the included Data Classes. ‘Type’ means that the data are split evenly by data associated with a particular type of data, where ‘Aro’ indicates Arousals, ‘Clean’ indicates data without any of the events, and ‘Event’ or ‘Other Event’ indicate any of the respiratory events or oximeter artifacts. ‘Target’ refers to what the ML model is trying to predict (e.g., arousals). A ‘Binary Target’, for example, is designed to determine if the Data Class is arousal or something else. “Num pClasses” means the number of predicted classes, i.e. the total number of Data Classes the ML Model is attempting to predict.

The term ‘Feature Set’ means the collection of features evaluated by the ML model. For ‘v1’ this includes: ‘sao2_mean’, ‘sao2_max’, ‘sao2_min’, ‘hr_mean’, ‘hr_max’, ‘hr_min’, ‘hrv_rmssd’, ‘hrv_mean_r2r’, ‘hrv_stdnn’, ‘hrv_nn50’, ‘hrv_pnn50’,‘hrv_max_r2r’, ‘hrv_min_r2r’, ‘rr’, ‘rr_max’, ‘rr_min’, ‘btbi_rmssd’, ‘btbi_rmssd_min’, ‘btbi_rmssd_max’, ‘btbi_var’, ‘btbi_var_max’, ‘btbi_var_min’, and ‘emg_tat’.

ML models labeled ‘v1+’ includes all these features, along with ‘sleep_stage’.

‘ML Model’ refers to the specific model used in AWS to make the calculations described in the table. Specifically, the following model types on AWS were trained and tested: LightGBM, CatBoost, XGBoost, Random Forest, Extra Trees, Linear Models, Neural networks implemented in Pytorch, Neural Networks implemented using fast.ai, and multilayer perceptron. In many cases, a weighted ensemble was used to achieve the best model performance.

‘Accuracy’ is the total number of annotations that the model correctly classifies divided by the total number of annotations, and is defined using the following equation, wherein ‘TP’ indicates ‘true positives’, ‘TN’ indicates ‘true negatives’, ‘FP’ indicates ‘false positives, and ‘FN’ indicates ‘false negatives’:

$$Accuracy = \frac{( T P + T N )}{( T P + T N + F P + F N )}$$

For calculations that used a weighted ensemble of ML models, the F1 score is used as a proxy for accuracy, and is defined as:

$$F 1 Score = \frac{2 \times ( Recall \times Precision )}{( Recall + Precision )}$$

$$where Recall = \frac{T P}{( T P + F N )}$$

$$and Precision = \frac{T P}{( T P + F P )}$$

Referring specifically to FIG. 9, model run 8, which processed all Data Classes, used a binary target and a weighted ensemble of ML models, yielded the best results using the SHHSD. The F1 score and AUC, calculated as described above, were 0.822 and 0.9 respectively. FIG. 10 shows the ROC plot for this particular model. Based on these results, this ML model appears to be ideal for calculating arousals and other Data Classes present in patients suffering from OSA.

During deployment of the SMK, the above-described ML model would typically operate on servers running in the cloud, as indicated by FIG. 2. As the SMK measures fresh data from the patient, the bedside hub collects it and transmits it to the cloud, where the model processes it to estimate various Data Classes. Results are then transmitted to the third-party software system in FIG. 2, where they can be analyzed and used, for example, to: 1) adjust the PAP therapy; 2) characterize a patient's health and progression towards a particular chronic disease (e.g., CHF); 3) used to replace out equipment (e.g., a leaky or aging PAP mask); or 4) for other applications directed towards improving a patient's outcome.

### 5. Related and Alternate Embodiments

In embodiments, the SMK ‘docks’ into a bedside hub than charges the SMK's internal Li:ion battery and simultaneously downloads and displays data. FIG. 11A and 11B show mechanical models of such a bedside hub 42; FIG. 11C shows a photograph of it connected through a hose to a PAP machine and docking the SMK. The bedside hub 42 includes a base 208 that is weighted to prevent the hub from tipping over, and houses a small-scale computing platform (not shown in the figure, but similar to the Raspberry Pi 4 as described in https://www.raspberrypi.eom/products/raspberry-pi-4-model-b/). The base 208 connects to a vertical supporting structure 209 that houses a 7″ touchpanel display 206. The touchpanel display 206 connects to the computing platform in the base 208 through a video cable, e.g., USB-C or HDMI. The touchpanel display can render a user interface, such as that shown in FIG. 12A-F and described below. The hub 42 also includes a neck 205 that connects to a mounting region 204 that, during charging, receives the mechanical housing 37 of the SMK. The mounting region 204 features a USB-C (male) connector that plugs into the USB-C (female) connector within the mounted on the PCB 13 and made available through an opening in the housing 13 (see, for example, component 29 in FIG. 5B). The USB-C (male) connector connects to the small-scale computing platform in the base 208 of the hub 42 through a cable. Once the SMK is plugged in to the hub 42, the USB-C (male) connector in the mounting region 204 supplies electrical current passed from the small-scale computing platform to the PCB 14 and through the cable, which is then used to charge the Li:ion battery 16. The cable also ports data collected from the patient, which is originally stored in Flash memory on the PCB 14, to the small-scale computing platform for follow-on analysis. Such analysis may include processing time-dependent waveforms collected from the patient as described above, or transmitted raw and/or processed data via wired or wireless means to a cloud-based system, as indicated in FIG. 2.

FIG. 12A-F show screen captures from a GUI that the touchpanel display renders during operation. The small-scale computing platform operates computer code that controls the GUI. As shown in FIG. 12A, in one embodiment, while the patient wears the SMK, the GUI renders the current time like a standard alarm clock. Software running on the small-scale computing platform periodically checks the USB port located in the mounting region 204 to determine if the SMK is plugged in. When it is, the GUI renders the screen shown in FIG. 12B, which poses questions for a simple survey, first asking about sleep quality, then mask comfort, and finally how many times the patient woke up during the night, as shown in FIG. 12D. When the survey is complete, the user is prompted to click a button (labeled ‘Click to Upload’ in FIG. 12B, 12D, and 12E) that collects data stored on flash memory on the SMK and sends it up to the cloud, as indicated in FIG. 2. When all data are uploaded, software operating on the cloud sends a packet back to the hub, indicating the upload process is complete and prompting the GUI to render the number of uploaded files, as shown in FIG. 12E.

Other surveys can be performed according to the invention. In embodiments, for example, these surveys can include simple games or puzzles that test the patient's mentation. Results from the surveys, particularly when coupled to physiological data collected by the SMK, can be used for ML and AI calculations and report generation, as shown for example in FIGS. 9 and 10.

As shown in FIG. 12C and 12F, the GUI can also render data (e.g., numerical and waveform data) that the SMK measures in real-time. The SMK typically transmits data to the bedside hub using Bluetooth®. In this mode, the combination of the SMK and hub can function similar to a conventional vital sign monitor in a hospital. For example, as shown in the figures, the GUI can render real-time ECG waveforms (FIG. 12C) and IPG waveforms (FIG. 12F), along with any other physiological parameter that the SMK measures.

Other embodiments are also within the scope of the invention. For example, as described above, ECG waveforms cannot always be measured with good signal-to-noise ratios from regions on the patient above the neck. However, as shown in FIG. 13A-13B, high-quality ECG waveforms can be measured from a first electrode within the SMK (indicated by circle 30b) contacting the patient 15 near the head or cheek, and a second ‘satellite’ electrode (indicated by circle 30a) contacting the patient 15 at any location on the chest. A thin cable 79 connects the electrodes indicated by circles 30a, 30b. Typically, in this embodiment, the first electrode in the SMK is a reusable electrode, such as one fabricated from conductive rubber or fabric; the second electrode is typically an adhesive electrode featuring a conductive hydrogel, rivet coated with an Ag:AgCl film, all supported by an adhesive backing. 3M Red Dot electrodes are such an example. In embodiments, the SMK includes a port (e.g., a port resembling a stereo-jack connector), and the thin cable 79 easily plugs in and out of the port. Such a configuration may be deployed, for example, if other HR-monitoring sensors on the SMK (e.g., the optical sensor measuring PPG waveforms, or the impedance sensor measuring IPG waveforms) indicate that the patient may be suffering from a cardiac arrhythmia. If such a condition is detected, the GUI prompts the patient to deploy the measurement condition indicated in FIG. 13A so that ECG waveforms—which are ideal for detecting cardiac arrhythmia—can be measured.

FIG. 13B shows a photograph of the GUI indicated in FIG. 12C (operating in this case on a mobile tablet computer) rendering an ECG waveform resulting from this configuration.

In other embodiments, signals from the wireless transceiver within the SMK can be analyzed (e.g., triangulated) to determine the patient's location. In this case, a computer operating at a central monitoring station, such as hub, can perform triangulation to determine the patient's location. In still other embodiments, the sensor can include a more conventional location system, such as a global positioning system (herein “GPS”). In embodiments, for example, the GPS and its associated antenna are typically included in the PCB within the SMK.

In related embodiments, the bedside hub may include a camera (e.g., a video camera) to record the patient during sleep. Outputs from the camera may be incorporated into the ML and AI models described above to better characterize the patient's sleep.

In other embodiments, the Li:ion battery within the SMK is charged through other means, such as wireless inductive charging. This would obviate charging based on USB-C, which requires an opening in the SMK's enclosure, thus making it susceptible to damage from ingressing fluids.

In still other embodiments, the SMK includes other sensors (e.g., optical and chemical sensors) for making enhanced measurements from the patient's breath. Such sensors, for example, can be used to augment results from the pressure sensor (e.g., the Bosch BME688 sensor) described above. Such a sensor can measure chemicals emitted from the patient's breath that are related to their glucose level and generally to their diabetes, e.g., actual glucose in the breath, or alternatively compounds such as acetone, β-hydroxybutyric acid, and acetoacetic acid, all of which can indicate diabetic ketoacidosis, a potentially life threatening condition for diabetic patients. As a particular example, the MQ138 sensor manufactured by the Zhengzhou Winsen Electronics Technology Co., Ltd. has shown efficacy in measuring acetone from human breath, which in turn has been shown to strongly correlate to conventional glucose measurements made using a glucometer (see, e.g., Salman et. al, “Blood Glucose Level Measurement from Breath Analysis”, World Academy of Science, Engineering and Technology, International Journal of Biomedical and Biological Engineering, Vol: 12, No: 9, 2018).

In other embodiments, the above-described sensors may also be used to characterize physical characteristics of the mask. For example, the microphone sensor may detect acoustic sounds that indicate that the mask is leaking or poorly fitting. The pressure sensor may detect similar things. The impedance electrodes connect to the actual mask, and may thus detect impedance properties of its silicone rubber that relate to mechanical properties, such as modulus of elasticity, flexibility, stiffness, etc. These parameters, in turn, may indicate if the mask is degrading. Optical sensors coupled to the mask and hose, and particularly the multi-frequency optical sensors described above, may be able to detect discoloration in these components and, like the impedance sensor, indicate that they may be degrading.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A system for monitoring a blood pressure value from a patient, comprising:
a wearable mask adapted to be coupled to a positive airway pressure (PAP) machine and configured to deliver a flow of gas generated by the PAP machine to an airway of a patient;
a first sensor connected directly to the wearable mask, the first sensor comprising an optical sensor configured to measure a time-dependent optical waveform from a first region underneath a first portion of the wearable mask, the time-dependent optical waveform comprising a first pulse;
a second sensor connected directly to the wearable mask, the second sensor comprising an impedance sensor configured to measure a time-dependent impedance waveform from a second region proximal to the wearable mask, the time-dependent impedance waveform comprising a second pulse; and,
a processing system attached to the wearable mask, the processing system comprising a microprocessor configured to: 1) receive digital representations of both the first and second pulses; 2) process the digital representations to determine a time difference between the first and second pulses, or a parameter calculated therefrom; and 3) process the time difference, or the parameter calculated therefrom, to determine the blood pressure value.

2. The system of claim 1, wherein the optical sensor comprises a first light source and a photodiode.

3. The system of claim 1, wherein the impedance sensor comprises at least one sense electrode, and at least one drive electrode.

4. The system of claim 4, wherein both the at least one sense electrode and at least one drive electrode comprise a conductive material selected from the following: rubber, polymer, fabric, metal, wire, mesh, and hydrogel.

5. The system of claim 4, wherein the at least one drive electrode is configured to inject a first electrical current into the second region.

6. The system of claim 5, wherein the impedance sensor is further configured to modulate the first electrical current at a frequency ranging from 5-500 kHz.

7. The system of claim 6, wherein the first electrical current has an amplitude ranging from 0.01-5 mA.

8. The system of claim 1, wherein the processing system is further configured to determine a foot of the first pulse and a foot of the second pulse.

9. The system of claim 8, wherein to determine the time difference between the first and second pulses, the processing system is further configured to calculate a temporal separation between at least one of the following features of the first pulse and the second pulse: foot, maximum slope of the pulse's rising edge, base, peak value, and rising edge.

10. The system of claim 1, wherein the processing system is further configured to calculate an inverse of the time difference between the first and second pulses.

11. The system of claim 10, wherein the processing system is further configured to process the inverse of the time difference between the first and second pulses with a calibration value to determine the blood pressure value.

12. The system of claim 1, further comprising a control system integrated directly into the wearable mask, the control system comprising the processing system.

13. A system for monitoring a blood pressure value from a patient, comprising:
a wearable mask;
a first sensor connected directly to the wearable mask, the first sensor comprising an optical sensor configured to measure a time-dependent optical waveform from a first region underneath a first portion of the wearable mask, the time-dependent optical waveform comprising a first pulse;
a second sensor connected directly to the wearable mask, the second sensor comprising an impedance sensor configured to measure a time-dependent impedance waveform from a second region proximal to the wearable mask, the time-dependent impedance waveform comprising a second pulse; and,
a processing system attached to the wearable mask, the processing system comprising a microprocessor configured to: 1) receive digital representations of both the first and second pulses; 2) process the digital representations to determine a time difference between the first and second pulses, or a parameter calculated therefrom; and 3) process the time difference, or the parameter calculated therefrom, to determine the blood pressure value.

14. The system of claim 13, wherein the optical sensor comprises a first light source and a photodiode.

15. The system of claim 13, wherein the impedance sensor comprises at least one sense electrode, and at least one drive electrode.
